SEQUENCE LISTING (30 sequences)

SEQ ID NO: 1 - DNA, 20 bp, Artificial Sequence
  Synthetic Primer
tctcggccaggatgaatttg

SEQ ID NO: 2 - DNA, 20 bp, Artificial Sequence
  Synthetic Probe
tttgaaggtggggcgcacga

SEQ ID NO: 3 - DNA, 20 bp, Artificial Sequence
  Synthetic Primer
cgctaccaccatttcttcga

SEQ ID NO: 4 - DNA, 20 bp, Artificial Sequence
  Synthetic Primer
aaacggctatctctggaagc

SEQ ID NO: 5 - DNA, 22 bp, Artificial Sequence
  Synthetic Probe
cccaccaccagcagacgaactt

SEQ ID NO: 6 - DNA, 20 bp, Artificial Sequence
  Synthetic Primer
tgtacttcttgttggcctcg

SEQ ID NO: 7 - DNA, 20 bp, Artificial Sequence
  Synthetic Primer
tgccccgttgagtattgatg

SEQ ID NO: 8 - DNA, 22 bp, Artificial Sequence
  Synthetic Probe
agcccccgtgataccagtacca

SEQ ID NO: 9 - DNA, 20 bp, Artificial Sequence
  Synthetic Primer
gcctgtagcttaacctgacc

SEQ ID NO: 10 - DNA, 20 bp, Artificial Sequence
  Synthetic Primer
aacagctaccaacgacagtc

SEQ ID NO: 11 - DNA, 24 bp, Artificial Sequence
  Synthetic Probe
tccactacgagaagtgctccagga

SEQ ID NO: 12 - DNA, 20 bp, Artificial Sequence
  Synthetic Primer
atcaaccgcaagaagagtgg

SEQ ID NO: 13 - DNA, 20 bp, Artificial Sequence
  Synthetic Primer
ataggaaaaacaggcgttgt

SEQ ID NO: 14 - DNA, 25 bp, Artificial Sequence
  Synthetic Probe
aggcaaagcatccatatctgcacga

SEQ ID NO: 15 - DNA, 20 bp, Artificial Sequence
  Synthetic Primer
accacaagcatgcattacat

SEQ ID NO: 16 - DNA, 20 bp, Artificial Sequence
  Synthetic Primer
cggcagaacgtcaggataaa

SEQ ID NO: 17 - DNA, 21 bp, Artificial Sequence
  Synthetic Probe
ggcagtgaggcagagattcca

SEQ ID NO: 18 - DNA, 20 bp, Artificial Sequence
  Synthetic Primer
atgcgcatcagccatattca

SEQ ID NO: 19 - DNA, 20 bp, Artificial Sequence
  Synthetic Primer
tgcgttttgatggtgcctat

SEQ ID NO: 20 - DNA, 20 bp, Artificial Sequence
  Synthetic Probe
cggtaccatcgcgcgacttt

SEQ ID NO: 21 - DNA, 20 bp, Artificial Sequence
  Synthetic Primer
caatccaacacggcatgaac

SEQ ID NO: 22 - DNA, 20 bp, Artificial Sequence
  Synthetic Primer
gtcgatcacgttgtagaagg

SEQ ID NO: 23 - DNA, 20 bp, Artificial Sequence
  Artificial Probe
tgcctgagcgcgaagggtat

SEQ ID NO: 24 - DNA, 20 bp, Artificial Sequence
  Synthetic Primer
gttctgtgtcgttgaaggaa

SEQ ID NO: 25 - DNA, 25 bp, Artificial Sequence
  Synthetic Probe
catttgttctggagcaggctgacgg

SEQ ID NO: 26 - DNA, 22 bp, Artificial Sequence
  Synthetic Primer
cgcgaagtcagagtcgacatag

SEQ ID NO: 27 - DNA, 20 bp, Artificial Sequence
  Synthetic Primer
aagacctcaacgccgatcac

SEQ ID NO: 28 - DNA, 20 bp, Artificial Sequence
  Synthetic Primer
gctttaagaaaagagcccgt

SEQ ID NO: 29 - DNA, 27 bp, Artificial Sequence
  Synthetic Probe
tgcatatcactcgctacaaaatgcact

SEQ ID NO: 30 - DNA, 20 bp, Artificial Sequence
  Synthetic Primer
cttctgctaaaaatggcggt

(19) United States
(12) Patent Application Publication
(10) Pub. No.: US 2021/0071172 A1
(43) Pub. Date: Mar. 11, 2021

(54) BACTERIAL CAPTURE SEQUENCING PLATFORM AND METHODS OF DESIGNING, CONSTRUCTING AND USING

(71) Applicant: THE TRUSTEES OF COLUMBIA UNIVERSITY IN THE CITY OF NEW YORK, New York, NY (US)

(72) Inventors: Walter Ian Lipkin, New York, NY (US); Orchid Allicock, New Haven, CT (US); Cheng Guo, Guangzhou (CN); Thomas Briese, White Plains, NY (US); Nischay Mishra, New York, NY (US)

(21) Appl. No.: 17/092,975

(22) Filed: Nov. 9, 2020

Related U.S. Application Data
(63) Continuation of application No. PCT/US2019/033922, filed on May 24, 2019.
(60) Provisional application No. 62/675,890, filed on May 24, 2018.
(60) Provisional application No. 62/724,104, filed on Aug. 29, 2018.

(51) Int. Cl. C12N 15/10; C12Q 1/6874

(52) U.S. Cl. CPC C12N 15/1093; C12Q 1/6874

(57) ABSTRACT

The present invention provides novel methods, systems, tools, and kits for the simultaneous detection, identification and/or characterization of pathogenic bacteria known or suspected to infect vertebrates, more specifically humans, as well as the detection, identification and/or characterization of antimicrobial resistant genes and biomarkers and the detection of novel bacteria and/or antimicrobial resistant genes. The methods, systems, tools, and kits described herein are based upon the bacterial capture sequencing platform (BacCapSeq), a novel platform developed by the inventors. The invention also provides methods of designing and constructing the bacterial capture sequencing platform.

### CROSS-REFERENCE TO OTHER APPLICATIONS

The present application claims priority to U.S. Patent Application Ser. Nos. 62/675,890, filed May 24, 2018 and 62/724,014, filed Aug. 29, 2018, both of which are hereby incorporated by reference in its entirety.

### STATEMENT OF GOVERNMENT SUPPORT

This invention was made with government support under AI109761 awarded by the National Institutes of Health. As such, the United States government has certain rights in this invention.

### FIELD OF THE INVENTION

This invention relates to the field of multiplex pathogenic bacteria detection, identification, and characterization using high throughput sequencing.

### BACKGROUND OF THE INVENTION

In the pre-antibiotic era, naturally occurring infectious disease was a common cause of mortality. For example, puerperal sepsis was a common cause of maternal mortality. Up to 30% of children did not survive their first year of life, and community acquired pneumonia and meningitis resulted in 30% and 70% mortality, respectively. The advent of bacterial diagnostics and antibiotics has not only reduced the burden of naturally occurring infectious diseases but has also enhanced our quality of life by enabling innovations in clinical medicine such as organ transplantation, joint replacement, and other invasive surgical procedures, immunosuppressive chemotherapy, and burn management. However, these advances are threatened by the emergence of antimicrobial resistance (AMR). In 2013, the collaborative World Economic Forum estimated 100,000 annual AMR-related deaths in the United States alone due to hospital-acquired infections (Golkar et al. 2014). The global impact of AMR is estimated at 700,000 deaths annually, with the highest burden in the developing world.

Early, accurate differential diagnosis of bacterial infections is critical to reducing morbidity, mortality, and health care costs. It can also reduce the inappropriate use of antibiotics. Multiplex PCR methods in common use for differential diagnosis of bacterial infections can identify potential pathogens but do not provide insights into the presence or expression of AMR genes. Furthermore, they do not include bacteria only rarely associated with significant disease, such as G. vaginalis, implicated here in unexplained sepsis in an individual with HIV/AIDS. Moreover, culture-based methods require two to several days to identify pathogens and even longer to provide antibiotic susceptibility profiles (Rhee et al. 2017). Accordingly, physicians typically administer broad-spectrum antibiotics pending acquisition of more specific information (Howell and Davis 2017).

No platform currently permits rapid and simultaneous insights into phylogeny, pathogenicity markers, and antimicrobial resistance needed to enable the early and precise antibiotic treatment that could reduce morbidity, mortality and economic burden.

Thus, there is a need for a sensitive cost-effective capture sequencing platform for the detection of pathogenic bacteria, especially in a clinical setting, as well as features associated with pathogenicity and antibiotic resistance. The current invention is a sensitive and specific high throughput (HTS)-based platform for clinical diagnosis and bacterial analysis of any type of sample.

### SUMMARY OF THE INVENTION

Described herein is a method for determining not only the bacterial composition of a sample but also the presence of features associated with pathogenicity and antibiotic resistance. The inventors have developed a pathogenic bacterial capture sequencing platform (BacCapSeq), which greatly enhances the sensitivity of sequence-based pathogenic bacteria detection and characterization. All known human bacterial pathogens are addressed as well as antimicrobial resistant genes. The platform was designed and constructed using 1.2 million protein coding sequences from 307 most important pathogenic bacterial species from the Pathosystems Resource Integration Center (PATRIC) database, along with all the known antimicrobial resistant genes from the Comprehensive Antibiotic Resistance Database (CARD), and virulence factors from the Virulence Factor Database (VFDB). These protein coding sequences were extracted and pooled together as the target sequences for capture. 4.2 million probes were designed (average probe length of 75 bp, average inter-probe spacing of 121 bp) to tile and cover relevant target sequences. A biotinylated oligonucleotide probe library containing those 4.2 million probes was used for solution-based capture of pathogenic bacterial nucleic acids present in complex samples containing variable proportions of different pathogenic bacterial and host nucleic acids. The use of BacCapSeq resulted in a 500 to 1,000-fold increase in bacterial reads from blood and cerebrospinal fluid, when compared to conventional Illumina sequencing.

The BacCapSeq platform is ideally suited for analyses of genome composition and dynamics and will enable transition of high throughput sequencing to clinical diagnostic as well as research applications.

The present invention provides novel methods, systems, tools, and kits for the simultaneous detection, identification and/or characterization of pathogenic bacteria known or suspected to infect vertebrates, in particular humans, as well as the presence of features associated with pathogenicity and antibiotic resistance. The methods, systems, tools, and kits described herein are based upon the bacterial capture sequencing platform (BacCapSeq), a novel platform developed by the inventors.

Accordingly, the present invention is a method of designing and/or constructing a bacterial capture sequencing platform utilizing a positive selection strategy for probes comprising nucleic acids derived from pathogenic bacteria as well as antimicrobial resistant genes, comprising the following steps.

The first step is to obtain sequence information from bacterial species, including but not limited to species known or suspected of being pathogenic to vertebrates, especially humans. Table 1 is a list of the 307 most important known pathogenic bacterial species.

The next step is extracting the coding sequences from the bacterial genomes. 1.2 million protein coding sequences from 307 of the most important known pathogenic bacterial species from the PATRIC database, along with all the known antimicrobial resistant genes from the CARD database and virulence factors from the VFDB database, were extracted and pooled together as the target sequences for capture.

In the next step, the coding sequences are broken into fragments of about 75 nucleotides (nt) in average length with a standard deviation of 5.8 nt. The probe melting temperature (Tm) is an average of about 82.7° C., with a standard deviation of about 5.7° C. (median melting temperature about 82.3° C., minimum melting temperature about 62.4° C. and maximum melting temperature about 100.7° C.).

Additionally, the fragments are tiled across the coding sequences in order to cover all sequences in a database with about 4.2 million probes which results in about 100 to about 150 nucleotides intervals with about 120 nucleotides being the average spacing or interval. If more probes are desired, the intervals can be smaller, less than about 50 nucleotides down to about 1 nucleotide, to even overlapping probes. If less probes are desired in the platform, the interval can be larger, about 150 to about 200 nucleotide intervals.

Embodiments of the present invention also provide automated systems and methods for designing and/or constructing the bacterial capture sequencing platform. Models made by the embodiments of the present invention may be used by persons in the art to design and/or construct a bacterial capture sequencing platform.

In some embodiments of the present invention, systems, apparatuses, methods, and computer readable media are provided that use bacterial and sequence information along with analytical tools in a design model for designing and/or constructing the bacterial capture sequencing platform. For example, in some embodiments, a first analytical tool comprising information from Table 1 disclosing bacterial species that include all known human pathogenic species can be used to find pertinent sequence information as well as all the known antimicrobial resistant genes from the Comprehensive Antibiotic Resistance Database (CARD) and virulence factors from the VFDB database and the pertinent sequence information processed using an algorithm to extract coding sequences and a second analytical tool to break the coding sequence into fragments for oligonucleotides with the proper parameters for the platform.

A further embodiment of the present invention is a novel platform otherwise known as the bacterial capture sequencing platform, designed and/or constructed using the methods described herein. In one embodiment, the platform comprises between about one million and about five million probes, preferably about four million probes. In one embodiment, the probes are oligonucleotide probes. In a further embodiment, the oligonucleotide probes are synthetic. The platform can comprise and/or derive from the genomes of pathogenic bacteria known or suspected to infect vertebrates, in particular humans, as well as antimicrobial resistant genes and virulence factors. In one embodiment, the probes of the platform comprise and/or derive from the genomes of pathogenic bacteria in Table 1. In a further embodiment, the probes of the platform can comprise and/or derive from genes from all the known antimicrobial resistant genes from the Comprehensive Antibiotic Resistance Database (CARD) and virulence factors from the Virulence Factor Database (VFDB). In one embodiment, the platform is in the form of an oligonucleotide probe library. In one embodiment, the oligonucleotides can comprise DNA, RNA, linked nucleic acids (LNA), bridged nucleic acids (BNA) or peptide nucleic acids (PNA) as well as any nucleic acids that can be derived naturally or synthesized now or in the future. In one embodiment the platform is in the form of a solution. In a further embodiment, the platform is in a solid-state form such as a microarray or bead. In a further embodiment, the oligonucleotides are modified by a composition to facilitate binding to a solid state.

One embodiment of the current invention is a database comprising information on the bacterial capture sequencing platform including at least the length, nucleotide sequence, melting temperature, and origin of each oligonucleotide probe. A further embodiment is computer-readable storage mediums with program code comprising information, e.g., a database, comprising information regarding the bacterial capture sequencing platform including at least the length, nucleotide sequence, melting temperature, and origin of each oligonucleotide probe.

Additionally, the present invention provides a method for constructing a sequencing library for the detection, identification and/or characterization of at least one bacterium or multiple bacteria using the bacterial capture sequencing platform in a positive selection scheme.

The present invention also provides systems for the simultaneous detection, identification and/or characterization of pathogenic bacteria and/or antimicrobial resistant genes or biomarkers, including those known and unknown, in any sample. The system includes at least one subsystem wherein the subsystem includes the bacterial capture sequencing platform of the invention. The system also can comprise subsystems for further detecting, identifying and/or characterizing of the bacteria, including but not limited to subsystems for preparation of the nucleic acids from the sample, hybridization, amplification, high throughput sequencing, and identification and characterization of the bacteria.

The present invention also provides methods for the simultaneous detection of bacteria and/or antimicrobial resistant genes or biomarkers in any sample utilizing the bacterial capture sequencing platform.

The present invention also provides methods for the simultaneous identification and characterization of bacteria and/or antimicrobial resistant genes or biomarkers in any sample utilizing the bacterial capture sequencing platform.

In some embodiments of the foregoing methods, more than one bacteria are detected, identified, and/or characterized. In some embodiments of the foregoing methods, more than ten bacteria are detected, identified, and/or characterized. In some embodiments of the foregoing methods, more than fifty bacteria are detected, identified, and/or characterized. In some embodiments of the foregoing methods, more than one hundred bacteria are detected, identified, and/or characterized. In some embodiments of the foregoing methods, more than one hundred and fifty bacteria are detected, identified, and/or characterized. In some embodiments of the foregoing methods, more than two hundred bacteria are detected, identified, and/or characterized. In some embodiments of the foregoing methods, more than two hundred and fifty bacteria are detected, identified, and/or characterized. In some embodiments of the foregoing methods, more than three hundred bacteria detected, identified, and/or characterized. In some embodiments of the foregoing methods, all pathogenic bacteria known or suspected to infect vertebrates are detected, identified, and/or characterized. In some embodiments of the foregoing methods, some or all of the bacteria listed in Table 1 are detected, identified, and/or characterized.

The present invention also provides for methods of detecting, identifying and/or characterizing unknown bacteria and/or antimicrobial resistant genes or biomarkers in any sample, utilizing the novel bacterial capture sequencing platform.

The present invention also provides for methods of detecting, identifying and/or characterizing AMR genes, both known and unknown in any sample, utilizing the novel bacterial capture sequencing platform.

A further embodiment is a kit for designing and/or constructing the bacterial capture sequencing platform comprising analytical tools to choose sequence information and break the coding sequences into fragments for oligonucleotides with the proper parameters for the platform.

A further embodiment is a kit for the detection, identification and/or characterization of pathogenic bacteria known or suspected to infect vertebrates and/or antimicrobial resistant genes or biomarkers comprising the bacterial capture sequencing platform and optionally primers, enzymes, reagents, and/or user instructions for the further detection, identification and/or characterization of at least one bacterium in a sample.

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

FIG. 1 shows that BacCapSeq yields more reads and higher genome coverage than unbiased high-throughput sequencing. FIG. 1A is a graphic representation of read depth obtained with BacCapSeq or unbiased high throughput sequencing (UHTS) across the K. pneumoniae genome. FIG. 1B is representative BacCapSeq results for the toxR virulence gene obtained from whole-blood nucleic acid spiked with 40,000 copies/ml of V. cholerae DNA. FIG. 1C is representative BacCapSeq results for the bla_{KPC} AMR gene obtained from whole blood spiked with 40,000 live K. pneumoniae cells/ml. In FIGS. 1B and 1C, probes are shown by the top lines, the BacCapSeq reads are shown in the middle lines and the UHTS reads are shown in the bottom lines.

FIG. 2 is a graph showing the mapped bacterial reads in blood spiked with bacterial cells. Mapped bacterial reads were normalized to 1 million quality- and host-filtered reads obtained by BacCapSeq (left hand bars) or UHTS (right hand bars). The data shown represent 40,000 cells/ml. No cutoff threshold was applied.

FIG. 3 shows the identification of bacteria in two immunosuppressed patients with HIV/AIDS and unexplained sepsis using BacCapSeq. FIG. 3A is a graph showing the identification of an infection with Salmonella enterica using BacCapSeq and UHTS. FIG. 3B is a graph showing the identification of a coinfection with Streptococcus pneumoniae and Gardnerella vaginalis using BacCapSeq and UHTS. FIG. 3C shows the genomic coverage of Gardnerella vaginalis using BacCapSeq and UHTS. The BacCapSeq resulted in a marked increase in percent of genome recovered.

FIG. 4 is a scatter plot showing the results of using BacCapSeq to detect antimicrobial resistance (AMR) biomarkers. Levels of seven transcripts in Staphylococcus aureus sensitive (AMR+) or resistant (AMR−) to ampicillin were measured after culture for 45, 90, and 270 minutes in the presence of ampicillin. Box plots represent the log of normalized transcript counts for each gene. Only results obtained with BacCapSeq are shown because no transcripts were detected in the presence of ampicillin with UHTS until later time points.

### DETAILED DESCRIPTION OF THE INVENTION

### Molecular Biology

In accordance with the present invention, there may be numerous tools and techniques within the skill of the art, such as those commonly used in molecular immunology, cellular immunology, pharmacology, and microbiology. See, e.g., Sambrook et al. (2001) Molecular Cloning: A Laboratory Manual. 3rd ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

As used herein and in the claims, the singular forms “a,” “an,” and “the” include the singular and the plural reference unless the context clearly indicates otherwise. Thus, for example, a reference to “an agent” includes a single agent and a plurality of such agents.

As used herein the terms “bacterial capture sequencing platform” and “BacCapSeq” will be used interchangeably and refer to the novel capture sequencing platform of the current invention that allows the simultaneous detection, identification and/or characterization of pathogenic bacteria known or suspected to infect vertebrates in any single sample in a single high throughput sequencing reaction. The terms denote the platform in every form, including but not limited to the collection of synthetic oligonucleotides representing the coding sequences of at least one pathogenic bacterium (i.e., “probe library”), either in solution or attached to a solid support, a database comprising information on the bacterial capture sequencing platform including at least the length, nucleotide sequence, melting temperature, and origin of each oligonucleotide probe, and computer-readable storage mediums with program code comprising information on the bacterial capture sequencing platform including at least the length, nucleotide sequence, melting temperature, and origin of each oligonucleotide probe.

The term “subject” as used in this application means an animal with an immune system such as avians and mammals. Mammals include canines, felines, rodents, bovine, equines, porcines, ovines, and primates. Avians include, but are not limited to, fowls, songbirds, and raptors. Thus, the invention can be used in veterinary medicine, e.g., to treat companion animals, farm animals, laboratory animals in zoological parks, and animals in the wild. The invention is particularly desirable for human medical applications.

The term “patient” as used in this application means a human subject.

The term “detection”, “detect”, “detecting” and the like as used herein means as used herein means to discover the presence or existence of.

The terms “identification”, “identify”, “identifying” and the like as used herein means to recognize a specific bacterium or bacteria and/or gene or genes in sample from a subject.

The term “characterization”, “characterize”, “characterizing” and the like as used herein means to describe or categorize by features, in some cases herein by sequence information.

As used herein, the term “isolated” and the like means that the referenced material is free of components found in the natural environment in which the material is normally found. In particular, isolated biological material is free of cellular components. In the case of nucleic acid molecules, an isolated nucleic acid includes a PCR product, an isolated mRNA, a cDNA, an isolated genomic DNA, or a restriction fragment. In another embodiment, an isolated nucleic acid is preferably excised from the chromosome in which it may be found. Isolated nucleic acid molecules can be inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated material may be, but need not be, purified.

As used herein, a “nucleic acid”, and “polynucleotide” and “nucleic acid sequence” and “nucleotide sequence” includes a nucleic acid, an oligonucleotide, a nucleotide, a polynucleotide, and any fragment, variant, or derivative thereof. The nucleic acid or polynucleotide may be double-stranded, single-stranded, or triple-stranded DNA or RNA (including cDNA), or a DNA-RNA hybrid of genetic or synthetic origin, wherein the nucleic acid contains any combination of deoxyribonucleotides and ribonucleotides and any combination of bases, including, but not limited to, adenine, thymine, cytosine, guanine, uracil, inosine, and xanthine hypoxanthine. As further used herein, the term “cDNA” refers to an isolated DNA polynucleotide or nucleic acid molecule, or any fragment, derivative, or complement thereof. It may be double-stranded, single-stranded, or triple-stranded, it may have originated recombinantly or synthetically, and it may represent coding and/or noncoding 5′ and/or 3′ sequences.

The term “fragment” when used in reference to a nucleotide sequence refers to portions of that nucleotide sequence. The fragments may range in size from 5 nucleotide residues to the entire nucleotide sequence minus one nucleic acid residue.

The term “genome” as used herein, refers to the entirety of an organism's hereditary information that is encoded in its primary DNA or RNA or nucleotide sequence (DNA or RNA as applicable). The genome includes both the genes and the non-coding sequences. For example, the genome may represent a viral genome, a microbial genome or a mammalian genome.

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

The term “sequencing library”, as used herein refers to a library of nucleic acids that are compatible with next-generation high throughput sequencers.

As used herein, the term “oligonucleotide” or “oligonucleotide probe” refers to a nucleic acid, generally of at least 10, preferably at least 15, and more preferably at least 20 nucleotides, preferably no more than 100 nucleotides, that is hybridizable to a genomic DNA molecule, a cDNA molecule, or an mRNA molecule encoding a gene, mRNA, cDNA, or other nucleic acid of interest. The nucleic acids that comprises the oligonucleotides include but are not limited to DNA, RNA, linked nucleic acids (LNA), bridged nucleic acids (BNA) and peptide nucleic acids (PNA). Oligonucleotides can be labeled, e.g., with ³²P-nucleotides or nucleotides to which a label, such as biotin, has been covalently conjugated.

The term “synthetic oligonucleotide” refers to single-stranded DNA or RNA molecules having preferably from about 10 to about 100 bases, which can be synthesized. In general, these synthetic molecules are designed to have a unique or desired nucleotide sequence, although it is possible to synthesize families of molecules having related sequences and which have different nucleotide compositions at specific positions within the nucleotide sequence. The term synthetic oligonucleotide will be used to refer to DNA or RNA molecules having a designed or desired nucleotide sequence.

The term “identifier” as used herein refers to any unique, non-naturally occurring, nucleic acid sequence that may be used to identify the originating genome of a nucleic acid fragment. The identifier function can sometimes be combined with other functionalities such as adapters or primers and can be located at any convenient position.

The terms “next-generation sequencing platform” and “high-throughput sequencing” and “HTS” as used herein, refer to any nucleic acid sequencing device that utilizes massively parallel technology. For example, such a platform may include, but is not limited to, Illumina sequencing platforms.

As used herein, the terms “complementary” or “complementarity” are used in reference to “polynucleotides” and “oligonucleotides” (which are interchangeable terms that refer to a sequence of nucleotides) related by the base-pairing rules. It may also include mimics of or artificial bases that may not faithfully adhere to the base-pairing rules. For example, the sequence “C-A-G-T,” is complementary to the sequence “G-T-C-A.” Complementarity can be “partial” or “total.” “Partial” complementarity is where one or more nucleic acid bases are not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

The term “nucleic acid hybridization” or “hybridization” refers to anti-parallel hydrogen bonding between two single-stranded nucleic acids, in which A pairs with T (or U if an RNA nucleic acid) and C pairs with G. Nucleic acid molecules are “hybridizable” to each other when at least one strand of one nucleic acid molecule can form hydrogen bonds with the complementary bases of another nucleic acid molecule under defined stringency conditions. Stringency of hybridization is determined, e.g., by (i) the temperature at which hybridization and/or washing is performed, and (ii) the ionic strength and (iii) concentration of denaturants such as formamide of the hybridization and washing solutions, as well as other parameters. Hybridization requires that the two strands contain substantially complementary sequences. Depending on the stringency of hybridization, however, some degree of mismatches may be tolerated. Under “low stringency” conditions, a greater percentage of mismatches are tolerable (i.e., will not prevent formation of an anti-parallel hybrid).

As used herein the term “hybridization product” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization product may be formed in solution or between one nucleic acid sequence present in solution and another nucleic acid sequence immobilized to a solid support.

As used herein, the term “Tₘ” is used in reference to the “melting temperature.” The melting temperature is the temperature at which a population of double-stranded nucleic acid molecules becomes half dissociated into single strands. As indicated by standard references, a simple estimate of the Tₘ value may be calculated by the equation: Tₘ=81.5+0.41 (% G+C), when a nucleic acid is in aqueous solution at 1M NaCl. Anderson et al., “Quantitative Filter Hybridization” In: Nucleic Acid Hybridization (1985). More sophisticated computations take structural, as well as sequence characteristics, into account for the calculation of Tₘ.

As used herein the term “stringency” is used in reference to the conditions of temperature, ionic strength, and the presence of other compounds such as organic solvents, under which nucleic acid hybridizations are conducted. “Stringency” typically occurs in a range from about Tₘ to about 20° C. to 25° C. below Tₘ. A “stringent hybridization” can be used to identify or detect identical polynucleotide sequences or to identify or detect similar or related polynucleotide sequences. For example, when fragments are employed in hybridization reactions under stringent conditions the hybridization of fragments which contain unique sequences (i.e., regions which are either non-homologous to or which contain less than about 50% homology or complementarity) are favored. Alternatively, when conditions of “weak” or “low” stringency are used hybridization may occur with nucleic acids that are derived from organisms that are genetically diverse (i.e., for example, the frequency of complementary sequences is usually low between such organisms).

“Amplification” is defined as the production of additional copies of a nucleic acid sequence and is generally carried out either in vivo, or in vitro, i.e. for example using polymerase chain reaction.

As used herein, the term “polymerase chain reaction” (“PCR”) refers to the method disclosed in U.S. Pat. Nos. 4,683,195 and 4,683,202, herein incorporated by reference, which describe a method for increasing the concentration of a segment of a target sequence in a mixture of genomic DNA without cloning or purification. The length of the amplified segment of the desired target sequence is determined by the relative positions of two oligonucleotide primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of 32P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications. With PCR, it is also possible to amplify a complex mixture (library) of linear DNA molecules, provided they carry suitable universal sequences on either end such that universal PCR primers bind outside of the DNA molecules that are to be amplified.

The terms “percent (%) sequence similarity”, “percent (%) sequence identity”, and the like, generally refer to the degree of identity or correspondence between different nucleotide sequences of nucleic acid molecules or amino acid sequences of proteins that may or may not share a common evolutionary origin. Sequence identity can be determined using any of a number of publicly available sequence comparison algorithms, such as BLAST, FASTA, DNA Strider, and GCG (Genetics Computer Group, Program Manual for the GCG Package, Version 7, Madison, Wis.).

To determine the percent identity between two amino acid sequences or two nucleic acid molecules, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are, or are about, of the same length. The percent identity between two sequences can be determined using techniques similar to those described below, with or without allowing gaps. In calculating percent sequence identity, typically exact matches are counted.

### The Bacterial Capture Sequencing Platform

Shown herein is a platform that increases the sensitivity of high-throughput sequencing for detection and characterization of bacteria, virulence determinants, and antimicrobial resistance (AMR) genes. The system uses a probe set comprised of 4.2 million oligonucleotides based on the Pathosystems Resource Integration Center (PATRIC) database, the Comprehensive Antibiotic Resistance Database (CARD), and the Virulence Factor Database (VFDB), representing 307 bacterial species that include all known human-pathogenic species, known antimicrobial resistant genes, and known virulence factors, respectively. The use of bacterial capture sequencing (BacCapSeq) resulted in an up to 1,000-fold increase in bacterial reads from blood samples and lowered the limit of detection by 1 to 2 orders of magnitude compared to conventional unbiased high-throughput sequencing (UHTS), down to a level comparable to that of agent-specific real-time PCR with as few as 5 million total reads generated per sample. It detected not only the presence of AMR genes but also biomarkers for AMR that included both constitutive and differentially expressed transcripts. The BacCapSeq platform is ideally suited for analyses of genome composition and dynamics and will enable transition of high throughput sequencing to clinical diagnostic as well as research applications.

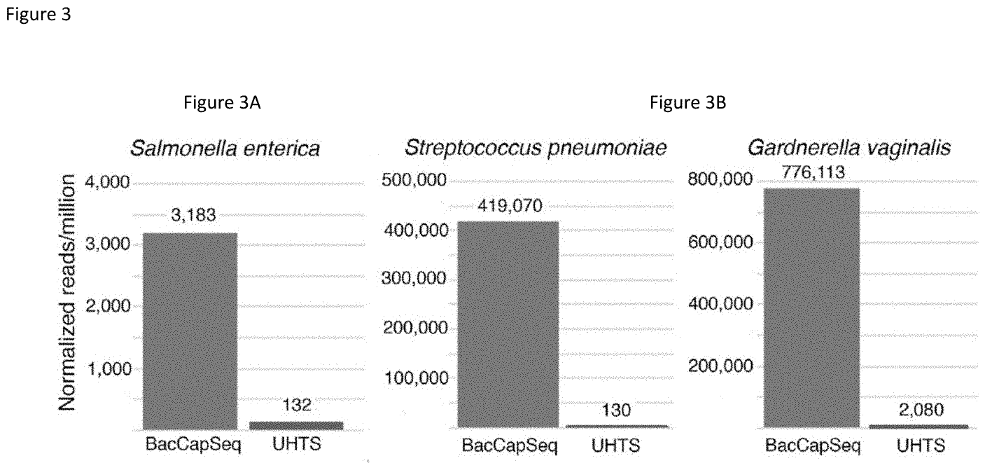

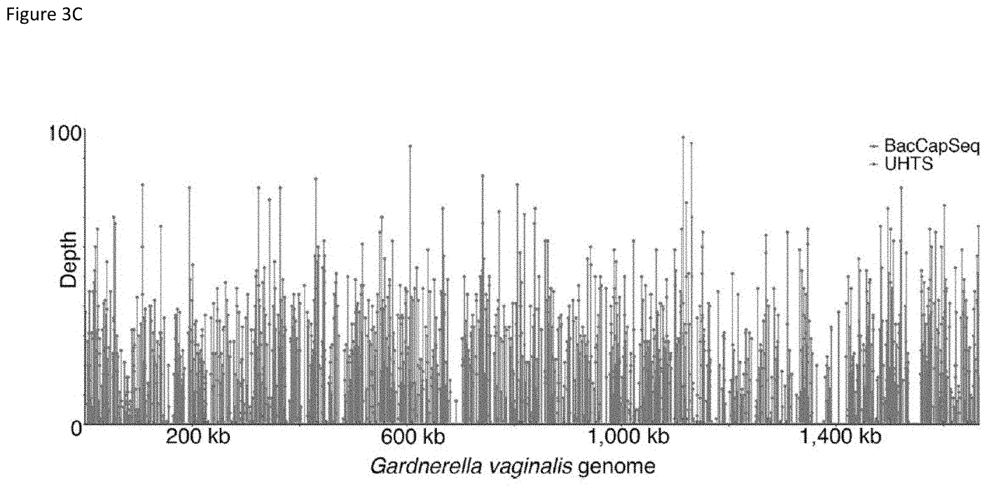

Results obtained with blood samples spiked with known concentrations of bacterial DNA (Example 3) or bacterial cells (Example 4) demonstrated a dose-dependent, consistent enhancement in the number of reads recovered and genome coverage obtained with BacCapSeq versus unbiased high throughput sequencing (UHTS). In instances where the bacterial load was as low as 40 cells per ml, UHTS detected no sequences of M. tuberculosis, K. pneumoniae, N. meningitidis, or S. pneumoniae and only one read for B. pertussis. In each of these instances, BacCapSeq detected multiple reads (M. tuberculosis, 6; K. pneumoniae, 522; N. meningitidis, 151; S. pneumoniae, 4; B. pertussis, 269) (Example 4; Table 4). This advantage was also observed in analysis of blood from patients with unexplained sepsis (Example 6; FIG. 3), where reads obtained were higher with BacCapSeq than UHTS for S. enterica (3,183 versus 132), S. pneumoniae (419,070 versus 130), and G. vaginalis (776,113 versus 2,080). These findings suggest that where levels of bacteria in blood are below 40 cells per ml, BacCapSeq has the potential to indicate the presence of a causal pathogen that might be missed by UHTS.

Incubation periods in blood culture systems commonly range from 3 days to 5 days (Bourbeau et al. 2005; Cockerill et al. 2004). Longer intervals may be required for sensitive detection of some pathogenic species of Neisseria, Rickettsia, Mycobacterium, Leptospira, Ehrlichia, Coxiella, Campylobacter, Burkholderia, Brucella, Bordetella, and Bartonella. An additional challenge is that bacterial loads may be low or intermittent. Cockerill et al. and Lee et al. have suggested that 80 ml of blood in four separate collections of at least 20 ml of blood are required for 99% test sensitivity in detecting viable bacteria. Current estimates of BacCapSeq sensitivity (a minimum of 40 copies per ml) corresponded favorably to the 80 ml sample volume recommended in culture tests (Lee et al. 2007). The American Society for Microbiology and the Clinical and Laboratory Standards Institute (CLSI) require false-positivity rates below 3% (CLSI 2007). Protocols for hygiene in diagnostic microbiology will be even more stringent with BacCapSeq than culture because nucleic acids are not eliminated by common disinfectants, thus decreasing false positives.

BacCapSeq also is designed to detect all AMR genes in the CARD database. Where these genes are located on bacterial chromosomes, it is anticipated that flanking sequences will allow association with specific bacteria within a sample, even when those samples contain more than one bacterial species. BacCapSeq will enable the discovery of constitutively expressed and induced transcripts that reflect the presence of functional bacterium-specific AMR elements.

The current invention includes a method of designing and/or constructing a bacterial capture sequencing platform, the platform itself, and methods of using the platform to construct sequencing libraries suitable for sequencing in any high throughput sequencing technology. The invention also includes methods and systems for simultaneously detecting pathogenic bacteria known or suspected to infect vertebrates, including humans, and/or antimicrobial resistant genes or biomarkers in a single sample, of any origin, using the novel bacterial capture sequencing platform. The present invention, denoted bacterial capture sequencing platform, or BacCapSeq, greatly enhances the sensitivity of sequence-based bacterial detection and characterization over current methods in the prior art. It enables detection of bacterial sequences in any complex sample backgrounds, including those found in clinical specimens. The invention allows the detection of bacterial composition of a sample but also the presence of features associated with pathogenicity and antibiotic resistance.

Accordingly, the present invention is a method of designing and/or constructing a sequence capture platform or technology otherwise known as bacterial capture sequencing platform or BacCapSeq. The present invention is a method of designing and/or constructing a sequence capture platform that comprises oligonucleotide probes selectively enriched for pathogenic bacteria and antimicrobial resistant genes, and the resulting bacterial capture sequencing platform. Accordingly, the method may include the following steps.

The first step is to obtain sequence information from pathogenic bacteria as well as antimicrobial resistant genes and virulence factors. In one embodiment, the bacteria listed in Table 1 are used for obtaining sequence data. In a further embodiment, new bacterium as well as newly discovered antimicrobial resistant genes can be included as well.

Sequence information is obtained from any public or private database of sequence information of bacteria and/or AMR genes and/or virulence factors, including but not limited to PATRIC, CARD and VFDB.

The second step of the method is to extract the coding sequences from the databases for use in designing the oligonucleotides.

Specifically, 1.2 million protein coding sequences from 307 important pathogenic bacterial species from the PATRIC database, along with all the known antimicrobial resistant genes from the CARD database, and virulence factors from the VFDB database, were extracted and pooled together as the target sequences for capture.

The next step of the method is to break the sequences into fragments to be the basis of the oligonucleotides. Specifically, about 4.2 million probes were designed with an average probe length of about 75 nt, and average inter-probe spacing of 121 nt to tile and cover all relevant target sequences.

The fragments are from about 50 to about 100 nucleotides in length, with about 75 nt being the average length, with a standard deviation of 5.8 nt (median length is about 75 nt, minimum length is about 50 nt, and maximum length is about 100 nt). The oligonucleotides can be refined as to length and start/stop positions as required by Tₘ and homopolymer repeats.

For example, the final Tₘ of the oligonucleotides should be similar and not too broad in range. The final Tₘ of the oligonucleotides in the exemplified platform ranged from about 62° C. to about 101° C., with about 82.7° C. being the average and a standard deviation of about 5.7° C. Thus, the fragment size can be adjusted accordingly to obtain oligonucleotides with the suitable melting temperatures.

Additionally, the fragments are tiled across the coding sequences in order to cover all sequences in a database with about 4.2 million probes which results in about 100 to about 150 nucleotides intervals with about 120 nucleotides being the average spacing. If more probes are desired, the intervals can be smaller, less than about 100 nucleotides down to about 1 nucleotide, to even overlapping probes. If less probes are desired in the platform, the interval can be larger, about 150 to about 200 nucleotides.

The present invention also relates to methods and systems that use computer-generated information to design and/or construct a bacterial capture sequencing platform. For example, in some embodiments, a first analytical tool using the information from Table 1 disclosing the pathogenic bacteria and all the known antimicrobial resistant genes from the Comprehensive Antibiotic Resistance Database (CARD) and virulence factors from the Virulence Factor Database (VFDB) can be used to find pertinent sequence information and the pertinent sequence information processed using an algorithm to extract coding sequences and a second analytical tool to fragment the coding sequences into oligonucleotides with the proper parameters for the platform including proper length, melting temperature, GC distribution, distance spaced between the oligonucleotides on the coding sequences, and percentage sequence identity.

In a further aspect of the present invention, analytical tools such as a first module configured to perform the choice of coding sequences from the bacteria in Table 1, all the known antimicrobial resistant genes from the Comprehensive Antibiotic Resistance Database (CARD) and virulence factors from the Virulence Factor Database (VFDB), and a second module to perform the fragmentation of the coding sequences may be provided that determines features of the oligonucleotides such as the proper length, melting temperature, GC distribution, distance spaced between the oligonucleotides on the coding sequences, and percentage sequence identity. The results of these tools form a model for use in designing the oligonucleotides for the bacterial capture sequencing platform.

An illustrative system for generating a design model includes an analytical tool such as a module configured to include bacteria from Table 1, all the known antimicrobial resistant genes from the Comprehensive Antibiotic Resistance Database (CARD), and virulence factors from the Virulence Factor Database (VFDB), and a database of sequence information. The analytical tool may include any suitable hardware, software, or combination thereof for determining correlations between the bacteria from Table 1 and the sequence data from database. A second analytical tool such as module is used to fragment the coding sequences. This analytical tool may include any suitable hardware, software, or combination for determining the necessary features of the oligonucleotides of the bacterial capture sequencing platform including proper length, melting temperature, GC distribution, distance spaced between the oligonucleotides on the coding sequences, and percentage sequence identity. In some embodiments of the invention, the features of the oligonucleotides are about 50 to 100 nucleotides in length, with a melting temperature ranging about 62° C. to about 101° C. and spaced at about 100 to 150 nucleotides intervals across coding sequences.

After the sequence information is obtained for the oligonucleotide probes, the oligonucleotides can be synthesized by any method known in the art including but not limited to solid-phase synthesis using phosphoramidite method and phosphoramidite building blocks derived from protected 2′-deoxynucleosides (dA, dC, dG, and T), ribonucleosides (A, C, G, and U), or chemically modified nucleosides, e.g. linked nucleic acids (LNA), bridged nucleic acids (BNA) or peptide nucleic acids (PNA).

The oligonucleotides can be refined as to length and start/stop positions as required by Tₘ and homopolymer repeats.

One embodiment of the platform is a library comprising the oligonucleotide probes that are capable of capturing nucleic acids from at least one pathogenic bacterium known or suspected to infect vertebrates. In some embodiments, the platform is a library comprising the oligonucleotide probes that are capable of capturing nucleic acids from more than one pathogenic bacteria known or suspected to infect vertebrates. In some embodiments, the platform is a library comprising the oligonucleotide probes that are capable of capturing nucleic acids from more than ten pathogenic bacteria known or suspected to infect vertebrates. In some embodiments, the platform is a library comprising the oligonucleotide probes that are capable of capturing nucleic acids from more than fifty pathogenic bacteria known or suspected to infect vertebrates. In some embodiments, the platform is a library comprising the oligonucleotide probes that are capable of capturing nucleic acids from more than one hundred pathogenic bacteria known or suspected to infect vertebrates. In some embodiments, the platform is a library comprising the oligonucleotide probes that are capable of capturing nucleic acids from more than one hundred and fifty pathogenic bacteria known or suspected to infect vertebrates. In some embodiments, the platform is a library comprising the oligonucleotide probes that are capable of capturing nucleic acids from more than two hundred pathogenic bacteria known or suspected to infect vertebrates. In some embodiments, the platform is a library comprising the oligonucleotide probes that are capable of capturing nucleic acids from more than two hundred and fifty pathogenic bacteria known or suspected to infect vertebrates. In some embodiments, the platform is a library comprising the oligonucleotide probes that are capable of capturing nucleic acids from more than three hundred pathogenic bacteria known or suspected to infect vertebrates. In some embodiments, the platform is a library comprising the oligonucleotide probes that are capable of capturing nucleic acids from the bacteria listed in Table 1.

A further embodiment is a library further comprising the oligonucleotide probes that are capable of capturing nucleic acids from AMR genes. A further embodiment is a library further comprising the oligonucleotide probes that are capable of capturing nucleic acids from virulence factors.

In one embodiment, the oligonucleotides of the platform are in solution.

In one embodiment of the present invention, the oligonucleotides comprising the bacterial capture sequencing platform are pre-bound to a solid support or substrate. Preferred solid supports include, but are not limited to, beads (e.g., magnetic beads (i.e., the bead itself is magnetic, or the bead is susceptible to capture by a magnet)) made of metal, glass, plastic, dextran (such as the dextran bead sold under the tradename, Sephadex (Pharmacia)), silica gel, agarose gel (such as those sold under the tradename, Sepharose (Pharmacia)), or cellulose); capillaries; flat supports (e.g., filters, plates, or membranes made of glass, metal (such as steel, gold, silver, aluminum, copper, or silicon), or plastic (such as polyethylene, polypropylene, polyamide, or polyvinylidene fluoride)); a chromatographic substrate; a microfluidics substrate; and pins (e.g., arrays of pins suitable for combinatorial synthesis or analysis of beads in pits of flat surfaces (such as wafers), with or without filter plates). Additional examples of suitable solid supports include, without limitation, agarose, cellulose, dextran, polyacrylamide, polystyrene, sepharose, and other insoluble organic polymers. Appropriate binding conditions (e.g., temperature, pH, and salt concentration) may be readily determined by the skilled artisan.

The oligonucleotides comprising the bacterial capture sequencing platform may be either covalently or non-covalently bound to the solid support. Furthermore, the oligonucleotides comprising the bacterial capture sequencing platform may be directly bound to the solid support (e.g., the oligonucleotides are in direct van der Waal and/or hydrogen bond and/or salt-bridge contact with the solid support), or indirectly bound to the solid support (e.g., the oligonucleotides are not in direct contact with the solid support themselves). Where the oligonucleotides comprising the bacterial capture sequencing platform are indirectly bound to the solid support, the nucleotides of the capture nucleic acid are linked to an intermediate composition that, itself, is in direct contact with the solid support.

To facilitate binding of the oligonucleotides comprising the bacterial capture sequencing platform to the solid support, the oligonucleotides comprising the bacterial capture sequencing platform may be modified with one or more molecules suitable for direct binding to a solid support and/or indirect binding to a solid support by way of an intermediate composition or spacer molecule that is bound to the solid support (such as an antibody, a receptor, a binding protein, or an enzyme). Examples of such modifications include, without limitation, a ligand (e.g., a small organic or inorganic molecule, a ligand to a receptor, a ligand to a binding protein or the binding domain thereof (such as biotin and digoxigenin)), an antigen and the binding domain thereof, an apatamer, a peptide tag, an antibody, and a substrate of an enzyme. In a preferred embodiment, the oligonucleotides comprise biotin.

Linkers or spacer molecules suitable for spacing biological and other molecules, including nucleic acids/polynucleotides, from solid surfaces are well-known in the art, and include, without limitation, polypeptides, saturated or unsaturated bifunctional hydrocarbons, and polymers (e.g., polyethylene glycol). Other useful linkers are commercially available.

In one embodiment of the present invention, a sequence of the oligonucleotides comprising the bacterial capture sequencing platform are the complement of (i.e., is complementary to) a sequence of the genome of at least one bacterium known or suspected to infect vertebrates as well as antimicrobial resistant genes and virulence factors. In another embodiment, the oligonucleotides comprising the bacterial capture sequencing platform are capable of hybridizing to a sequence of the genome of at least one bacterium known or suspected to infect vertebrates as well as antimicrobial resistant genes and virulence factors under stringent conditions.

In a further embodiment of the present invention, a sequence of the oligonucleotides comprising the bacterial capture sequencing platform are the complement of (i.e., is complementary to) a sequence of the genome of more than one pathogenic bacteria known or suspected to infect vertebrates as well as antimicrobial resistant genes and virulence factors. In another embodiment, the oligonucleotides comprising the bacterial capture sequencing platform are capable of hybridizing to a sequence of the genome of more than one pathogenic bacteria known or suspected to infect vertebrates as well as antimicrobial resistant genes and virulence factors under stringent conditions.

In a further embodiment of the present invention, a sequence of the oligonucleotides comprising the bacterial capture sequencing platform are the complement of (i.e., is complementary to) a sequence of the genome of more than fifty pathogenic bacteria known or suspected to infect vertebrates as well as antimicrobial resistant genes and virulence factors. In another embodiment, the oligonucleotides comprising the bacterial capture sequencing platform are capable of hybridizing to a sequence of the genome of more than fifty pathogenic bacteria known or suspected to infect vertebrates as well as antimicrobial resistant genes and virulence factors under stringent conditions.

In a further embodiment of the present invention, a sequence of the oligonucleotides comprising the bacterial capture sequencing platform are the complement of (i.e., is complementary to) a sequence of the genome of more than one hundred pathogenic bacteria known or suspected to infect vertebrates as well as antimicrobial resistant genes and virulence factors. In another embodiment, the oligonucleotides comprising the bacterial capture sequencing platform are capable of hybridizing to a sequence of the genome of more than one hundred pathogenic bacteria known or suspected to infect vertebrates as well as antimicrobial resistant genes and virulence factors under stringent conditions.

In a further embodiment of the present invention, a sequence of the oligonucleotides comprising the bacterial capture sequencing platform are the complement of (i.e., is complementary to) a sequence of the genome of more than one hundred and fifty pathogenic bacteria known or suspected to infect vertebrates as well as antimicrobial resistant genes and virulence factors. In another embodiment, the oligonucleotides comprising the bacterial capture sequencing platform are capable of hybridizing to a sequence of the genome of more than one hundred and fifty pathogenic bacteria known or suspected to infect vertebrates as well as antimicrobial resistant genes and virulence factors under stringent conditions.

In a further embodiment of the present invention, a sequence of the oligonucleotides comprising the bacterial capture sequencing platform are the complement of (i.e., is complementary to) a sequence of the genome of more than two hundred pathogenic bacteria known or suspected to infect vertebrates as well as antimicrobial resistant genes and virulence factors. In another embodiment, the oligonucleotides comprising the bacterial capture sequencing platform are capable of hybridizing to a sequence of the genome of more than two hundred pathogenic bacteria known or suspected to infect vertebrates as well as antimicrobial resistant genes and virulence factors under stringent conditions.

In a further embodiment of the present invention, a sequence of the oligonucleotides comprising the bacterial capture sequencing platform are the complement of (i.e., is complementary to) a sequence of the genome of more than two hundred and fifty pathogenic bacteria known or suspected to infect vertebrates as well as antimicrobial resistant genes and virulence factors. In another embodiment, the oligonucleotides comprising the bacterial capture sequencing platform are capable of hybridizing to a sequence of the genome of more than two hundred and fifty pathogenic bacteria known or suspected to infect vertebrates as well as antimicrobial resistant genes and virulence factors under stringent conditions.

In a further embodiment of the present invention, a sequence of the oligonucleotides comprising the bacterial capture sequencing platform are the complement of (i.e., is complementary to) a sequence of the genome of more than three hundred pathogenic bacteria known or suspected to infect vertebrates as well as antimicrobial resistant genes and virulence factors. In another embodiment, the oligonucleotides comprising the bacterial capture sequencing platform are capable of hybridizing to a sequence of the genome of more than three hundred pathogenic bacteria known or suspected to infect vertebrates as well as antimicrobial resistant genes and virulence factors under stringent conditions.

In a further embodiment of the present invention, a sequence of the oligonucleotides comprising the bacterial capture sequencing platform are the complement of (i.e., is complementary to) a sequence of the genome of some or all of the bacteria listed in Table 1 as well as antimicrobial resistant genes and virulence factors. In another embodiment, the oligonucleotides comprising the bacterial capture sequencing platform are capable of hybridizing to a sequence of the genome of some of all of the bacteria listed in Table 1 as well as antimicrobial resistant genes and virulence factors under stringent conditions.

The “complement” of a nucleic acid sequence refers, herein, to a nucleic acid molecule which is completely complementary to another nucleic acid, or which will hybridize to the other nucleic acid under conditions of high stringency. High-stringency conditions are known in the art. See, e.g., Maniatis et al., Molecular Cloning: A Laboratory Manual, 2nd ed. (Cold Spring Harbor: Cold Spring Harbor Laboratory, 1989) and Ausubel et al., eds., Current Protocols in Molecular Biology (New York, N.Y.: John Wiley & Sons, Inc., 2001). Stringent conditions are sequence-dependent, and may vary depending upon the circumstances.

In the exemplified embodiment, the oligonucleotides comprising the bacterial capture sequencing platform are synthesized using a cleavable programmable array wherein the array comprises the oligonucleotides comprising the bacterial capture sequencing platform. The oligonucleotides are cleaved from the array and hybridized with the nucleic acids from the sample in solution.

The present invention also includes the sequence capture platform otherwise known as bacterial capture sequencing platform made from one method of the invention. The platform comprises about 4.2 million probes. The oligonucleotides comprise sequences derived from the genomes of the bacteria listed in Table 1 as well as sequences derived from antimicrobial resistant genes and virulence factors.

The bacterial capture sequencing platform of the present invention can be in the form of a collection of oligonucleotides, preferably designed as set forth above, i.e., a probe library. The oligonucleotides can be in solution or attached to a solid state, such as an array or a bead. Additionally, the oligonucleotides can be modified with another molecule. In a preferred embodiment, the oligonucleotides comprise biotin.

The bacterial capture sequencing platform can also be in the form of a database or databases which can include information regarding the sequence and length and Tₘ of each oligonucleotide probe, and the bacterium from which the oligonucleotide sequence derived as well as antimicrobial resistant genes and virulence factors. The database can searchable. From the database, one of skill in the art can obtain the information needed to design and synthesis the oligonucleotide probes comprising the bacterial capture sequencing platform. The databases can also be recorded on machine-readable storage medium, any medium that can be read and accessed directly by a computer. A machine-readable storage medium can comprise, for example, a data storage material that is encoded with machine-readable data or data arrays. Machine-readable storage medium can include but are not limited to magnetic storage media, optical storage media, electrical storage media, and hybrids. One of skill in the art can easily determine how presently known machine-readable storage medium and future developed machine-readable storage medium can be used to create a manufacture of a recording of any database information. “Recorded” refers to a process for storing information on a machine-readable storage medium using any method known in the art.

TABLE 1
Bacteria targeted in BacCapSeq

| | | | Genome | CDS |
|---|---|---|---|---|
| GenomeID | Species Name | Strain Name | Length | Length |
| 1325130.3 | Helicobacter fennelliae | MRY12-0050 | 2155647 | 1928889 |
| 1313.7035 | Streptococcus pneumoniae | strain 225994 | 2473562 | 2156347 |
| 342451.11 | Staphylococcus saprophyticus | subsp. saprophyticus | 2577899 | 2141946 |
| | | ATCC 15305 | | |
| 13690.22 | Sphingobium yanoikuyae | strain B2 | 5901687 | 5313993 |
| 1403312.3 | Lactobacillus gasseri | 130918 | 1955817 | 1747071 |
| 521006.8 | Neisseria gonorrhoeae | NCCP11945 | 2236178 | 1859739 |
| 243275.7 | Treponema denticola | ATCC 35405 | 2843201 | 2585469 |
| 1648.207 | Erysipelothrix rhusiopathiae | strain GXBY-1 | 1876490 | 1675233 |
| 83554.68 | Chlamydia psittaci | strain Ho Re lower | 1239672 | 1126943 |
| 1408887.3 | Brucella canis | str. Oliveri | 3318660 | 2851011 |
| 553177.6 | Capnocytophaga sputigena | ATCC 33612 | 2988915 | 2640117 |
| 470.1295 | Acinetobacter baumannii | strain AB30 | 4335793 | 3827520 |
| 941429.3 | Shigella dysenteriae | CDC 74-1112 | 4592898 | 3898374 |
| 1138937.3 | Enterococcus faecium | EnGen0375 | 3073033 | 2588811 |
| | | [PRJNA206264] | | |
| 997885.3 | Bacteroides ovatus | CL02T12C04 | 7877545 | 7074510 |
| 469610.4 | Burkholderiales bacterium | 1_1_47 | 2643265 | 2267589 |
| 550773.4 | Ureaplasma urealyticum | serovar 9 str. ATCC | 947165 | 854097 |
| | | 33175 | | |
| 272831.7 | Neisseria meningitidis | FAM18 | 2194961 | 1886319 |
| 1206721.4 | Nocardia asiatica | NBRC 100129 | 8396852 | 7019652 |
| 469378.5 | Cryptobacterium curtum | DSM 15641 | 1617804 | 1379547 |
| 545774.3 | Streptococcus gallolyticus | subsp. gallolyticus | 2239771 | 1956687 |
| | | TX20005 | | |
| 1381751.3 | Brevibacterium sp. | VCM10 | 3844920 | 3423168 |
| 1073999.4 | Cronobacter condimenti | 1330 | 4456592 | 3858804 |
| 1191522.3 | Vibrio harveyi | ZJ0603 | 6626696 | 5594151 |
| 1158614.4 | Enterococcus gilvus | ATCC BAA-350 | 4179913 | 3613452 |
| | | [PRJNA206359] | | |
| 211110.3 | Streptococcus agalactiae | NEM316 | 2211485 | 1957587 |
| 1150423.6 | Bifidobacterium dentium | JCM 1195 = DSM | 2668067 | 2361810 |
| | | 20436 | | |
| 441157.9 | Burkholderia thailandensis | MSMB43 | 7245989 | 6466938 |
| 1504.11 | Clostridium septicum | strain P1044 | 3298970 | 2854944 |
| 1334630.3 | Enterobacter cloacae | EC 38VIM1 | 5140210 | 4496121 |
| 272947.5 | Rickettsia prowazekii | str. Madrid E | 1111523 | 850581 |
| 818.4 | Bacteroides thetaiotaomicron | strain 14-106904-2 | 6554963 | 5954626 |
| 87883.44 | Burkholderia multivorans | strain D2095 | 6668882 | 5957769 |
| 1005999.3 | Leminorella grimontii | ATCC 33999 | 4217979 | 3597366 |
| 1190567.3 | Stenotrophomonas | EPM1 | 9567626 | 8372517 |
| | maltophilia | | | |
| 1242968.3 | Campylobacter concisus | UNSWCS | 2072911 | 1858716 |
| 1661.14 | Trueperella pyogenes | strain 1117_TPYO | 4339061 | 3916941 |
| 216594.6 | Mycobacterium marinum | M | 6660144 | 5939325 |
| 272633.4 | Mycoplasma penetrans | HF-2 | 1358633 | 1193352 |
| 991936.4 | Vibrio cholerae | HC-81A1 | 4084020 | 3545079 |
| 47466.3 | Borrelia miyamotoi | CT14D4 | 907293 | 836034 |
| 1450190.3 | Streptococcus uberis | 6780 | 1960858 | 1774536 |
| 827.3 | Campylobacter ureolyticus | strain CIT007 | 1665702 | 1533513 |
| 547045.3 | Neisseria sicca | ATCC 29256 | 2824960 | 2274387 |
| 527012.3 | Yersinia kristensenii | ATCC 33638 | 5023212 | 4295709 |
| 226185.9 | Enterococcus faecalis | V583 | 3359974 | 2914284 |
| 1715020.3 | Enterobacter sp. | HMSC055A11 | 5771047 | 5147646 |
| 717608.3 | Clostridium cf. | saccharolyticum K10 | 3769775 | 3100935 |
| 243273.25 | Mycoplasma genitalium | G37 | 580076 | 550602 |
| 1234597.4 | Ochrobactrum intermedium | M86 | 5174353 | 4455606 |
| 1170698.3 | Rhodococcus sp. | R1101 | 4498032 | 3721392 |
| 283166.5 | Bartonella henselae | str. Houston-1 | 1931047 | 1462377 |
| 1302.34 | Streptococcus gordonii | strain FSS3 | 2308242 | 2053659 |
| 445970.5 | Alistipes putredinis | DSM 17216 | 2547410 | 2030679 |
| 521000.6 | Providencia rettgeri | DSM 1131 | 4747235 | 3833925 |
| 1675902.3 | Acinetobacter sp. | VT 511 | 3416321 | 2909631 |
| 336982.7 | Mycobacterium tuberculosis | F11 | 4424435 | 4010607 |
| 1331279.3 | Bordetella pertussis | CHOC0019 | 4149726 | 3710577 |
| 43675.28 | Rothia mucilaginosa | strain NUM-Rm6536 | 2292716 | 1909845 |
| 1363.18 | Lactococcus garvieae | M14 | 2253704 | 1964049 |
| 401472.3 | Corynebacterium | strain IMMIB RIV- | 2328280 | 2063352 |
| | ureicelerivorans | 2301 | | |
| 246432.29 | Staphylococcus equorum | strain 738_7 | 3070780 | 2602473 |
| 484.5 | Neisseria flavescens | strain CD-NF2 | 2345024 | 2060904 |
| 742729.3 | Bifidobacterium animalis | subsp. lactis Bi-07 | 1938822 | 1667571 |
| 398577.6 | Burkholderia ambifaria | MC40-6 | 7642536 | 6484158 |
| 546268.4 | Neisseria subflava | NJ9703 | 2272049 | 1942728 |
| 500638.3 | Edwardsiella tarda | ATCC 23685 | 3701950 | 2893728 |
| 568814.3 | Streptococcus suis | BM407 | 2170808 | 1886871 |
| 596328.3 | Mobiluncus mulieris | 28-1 | 2444798 | 2080260 |
| 1267000.5 | Mycoplasma hominis | ATCC 27545 | 715165 | 649725 |
| 1309.88 | Streptococcus mutans | strain AD01 | 2066006 | 1808274 |
| 515608.9 | Ureaplasma parvum | serovar 1 str. ATCC | 753674 | 687795 |
| | | 27813 | | |
| 283165.4 | Bartonella quintana | str. Toulouse | 1581384 | 1178793 |
| 445974.6 | Clostridium ramosum | DSM 1402 | 3235195 | 2840595 |
| 714315.3 | Leptotrichia goodfellowii | DSM 19756 | 2280962 | 2057127 |
| 748003.8 | Vibrio vulnificus | VVyb1(BT3) | 10784829 | 9391059 |
| 340100.3 | Bordetella petrii | DSM 12804 | 5287950 | 4596405 |
| 32022.148 | Campylobacter jejuni | subsp. jejuni strain | 1831013 | 1719324 |
| | | 00-0949 | | |
| 1339342.3 | Parabacteroides distasonis | str. 3776 D15 i | 5788520 | 5056515 |
| 272944.4 | Rickettsia conorii | str. Malish 7 | 1268755 | 1031538 |
| 85698.16 | Achromobacter xylosoxidans | strain MN001 | 5876049 | 5285721 |
| 764291.3 | Streptococcus urinalis | 2285-97 | 2145755 | 1886991 |
| 59201.158 | Salmonella enterica | subsp. enterica strain | 5190370 | 4587375 |
| | | YU39 | | |
| 471881.3 | Proteus penneri | ATCC 35198 | 3747952 | 3053205 |
| 500639.8 | Enterobacter cancerogenus | ATCC 35316 | 4635488 | 4062045 |
| 1041522.3 | Mycobacterium colombiense | CECT 3035 | 5573201 | 5049537 |
| 218496.4 | Tropheryma whipplei | TW08/27 | 925938 | 809589 |
| 519441.6 | Streptobacillus moniliformis | DSM 12112 | 1673280 | 1499988 |
| 1189613.3 | Staphylococcus massiliensis | CCUG 55927 | 2318102 | 1927416 |
| 931437.3 | Staphylococcus aureus | subsp. aureus | 3067858 | 2541390 |
| | | CIG1500 | | |
| 300.12 | Pseudomonas mendocina | strain 1267_PMEN | 6737888 | 6084486 |
| 1370127.3 | Legionella pneumophila | Leg01/16 | 3622637 | 2996880 |
| 29461.1 | Brucella suis | strain ZW046 | 3493280 | 3023487 |
| 386894.6 | Streptococcus iniae | 9117 | 2078160 | 1852968 |
| 1736395.3 | Arthrobacter sp. | Soil736 | 5887135 | 5154267 |
| 1197719.3 | Salmonella bongori | N268-08 | 4773537 | 4175097 |
| 479437.5 | Eggerthella lenta | DSM 2243 | 3632260 | 3114063 |
| 471874.6 | Providencia stuartii | ATCC 25827 | 4596738 | 3742128 |
| 1262908.3 | Mycoplasma sp. | CAG: 956 | 1442272 | 1289904 |
| 176279.9 | Staphylococcus epidermidis | RP62A | 2643840 | 2198358 |
| 428126.7 | Clostridium spiroforme | DSM 1552 | 2507885 | 2168592 |
| 76860.6 | Streptococcus constellatus | 925_SCON | 2043273 | 1822344 |
| 670.961 | Vibrio parahaemolyticus | strain FORC_023 | 5015214 | 4337505 |
| 992065.3 | Helicobacter pylori | Hp H-18 | 1759874 | 1588575 |
| 1193128.3 | Parascardovia denticolens | IPLA 20019 | 1995225 | 1692231 |
| 796945.3 | Oribacterium sp. | ACB8 | 2481911 | 2189736 |
| 1194086.3 | Yersinia enterocolitica | subsp. enterocolitica | 4518498 | 3833265 |
| | | WA-314 | | |
| 1719.1363 | Corynebacterium | strain 39 | 2403579 | 2124336 |
| | pseudotuberculosis | | | |
| 553218.4 | Campylobacter rectus | RM3267 | 2496160 | 2110443 |
| 747.324 | Pasteurella multocida | strain NIVEDI/PMS- | 2543931 | 2268661 |
| | | 1 | | |
| 1212545.3 | Staphylococcus arlettae | CVD059 | 2562113 | 2151681 |
| 1299326.3 | Mycobacterium kansasii | 662 | 6896162 | 6062763 |
| 992012.3 | Vibrio sp. | HENC-03 | 5881862 | 5062686 |
| 596318.3 | Acinetobacter radioresistens | SK82 | 3274578 | 2770728 |
| 649742.3 | Actinomyces odontolyticus | F0309 | 2430527 | 2007258 |
| 355276.3 | Leptospira borgpetersenii | serovar Hardjo-bovis | 3931782 | 3237096 |
| | | str. L550 | | |
| 562983.3 | Gemella sanguinis | M325 | 1747214 | 1489983 |
| 864569.5 | Streptococcus bovis | ATCC 700338 | 2077360 | 1767708 |
| 1175313.3 | Rickettsia honei | RB | 1268758 | 1026309 |
| 342113.3 | Burkholderia oklahomensis | strain EO147 | 7313670 | 6258960 |
| 1172204.3 | Clostridium sordellii | 8483 | 7613862 | 6043227 |
| 1206729.4 | Nocardia exalbida | NBRC 100660 | 7337483 | 6346974 |
| 1882747.3 | Afipia sp. | GAS231 | 7584236 | 6631098 |
| 1140002.3 | Enterococcus avium | ATCC 14025 | 4619322 | 3971613 |
| 222.8 | chromobacter undefined | 7393 | 6891463 | 6041772 |
| 1431713.3 | Pseudomonas aeruginosa | VRFPA07 | 7177216 | 6226170 |
| 257309.4 | Corynebacterium diphtheriae | NCTC 13129 | 2488635 | 2168952 |
| 83558.18 | Chlamydia pneumonia | UNKNOWN | 1229887 | 1112265 |
| 1299332.3 | Mycobacterium ulcerans | str. Harvey | 6247430 | 5197422 |
| 1681.46 | Bifidobacterium bifidum | strain 85B | 2360966 | 2051940 |
| 208962.32 | Escherichia albertii | strain K7394 | 5120257 | 4529373 |
| 873517.3 | Capnocytophaga ochracea | F0287 | 2655842 | 2267472 |
| 269484.6 | Ehrlichia canis | str. Jake | 1315030 | 952644 |
| 434924.5 | Coxiella burnetii | CbuK_Q154 | 2102380 | 1821327 |
| 1230476.3 | Bradyrhizobium sp. | DFCI-1 | 7645871 | 6517140 |
| 216816.113 | Bifidobacterium longum | strain 981_BLON | 3121288 | 2704191 |
| 71999.8 | Kocuria palustris | strain W4 | 3085907 | 2741640 |
| 1208591.3 | Cronobacter malonaticus | 681 | 4520983 | 3367032 |
| 904338.3 | Staphylococcus warneri | VCU121 | 2441494 | 2038356 |
| 28131.4 | Prevotella intermedia | strain 17-2 | 2737273 | 2386833 |
| 470735.4 | Brucella inopinata | BO1 | 3355593 | 2929914 |
| 1188238.3 | Mycoplasma capricolum | subsp. capricolum | 1032230 | 915789 |
| | | 14232 | | |
| 557598.3 | Laribacter hongkongensis | HLHK9 | 3169329 | 2678031 |
| 1267754.3 | Corynebacterium urealyticum | DSM 7111 | 2316065 | 2009727 |
| 203275.8 | Tannerella forsythia | ATCC 43037 | 3405521 | 2992134 |
| 303.188 | Pseudomonas putida | strain | 6958027 | 6169482 |
| | | FDAARGOS_121 | | |
| 813.62 | Chlamydia trachomatis | strain H17IMS | 18778151 | 16345362 |
| 445336.4 | Clostridium botulinum | Bf | 4194816 | 3373134 |
| 758847.3 | Leptospira santarosai | serovar Shermani str. | 3874350 | 3339084 |
| | | LT 821 | | |
| 932676.3 | Shigella boydii | ATCC 9905 | 5127771 | 4404261 |
| 216599.7 | Shigella sonnei | 53G | 5179725 | 4383876 |
| 883081.3 | Alloiococcus otitis | ATCC 51267 | 1776951 | 1516857 |
| 1689868.3 | Shewanella sp. | Sh95 | 4820870 | 4182549 |
| 883092.3 | Lactobacillus crispatus | FB077-07 | 2519002 | 2174664 |
| 349747.9 | Yersinia pseudotuberculosis | IP 31758 | 4935125 | 4148253 |
| 1441736.4 | Fusobacterium necrophorum | BFTR-2 | 2608490 | 2152095 |
| 306264.5 | Campylobacter upsaliensis | RM3195 | 1773834 | 1653024 |
| 1074132.3 | Streptococcus sobrinus | TCI-157 | 6599903 | 4512978 |
| 527019.3 | Bacillus thuringiensis | IBL 200 | 6731790 | 5431932 |
| 1348244.3 | Kingella kingae | KK245 | 1849366 | 1588950 |
| 765063.3 | Propionibacterium acnes | HL099PA1 | 2562711 | 2254332 |
| 1416915.5 | Aeromonas hydrophila | NJ-35 | 5279644 | 4641681 |
| 649743.3 | Actinomyces sp. | oral taxon 848 str. | 2519868 | 2082282 |
| | | F0332 | | |
| 37734.13 | Enterococcus casseliflavus | strain NLAE-zl-G268 | 3686667 | 3242505 |
| 28450.15 | Burkholderia pseudomallei | strain QCMRI_BP07 | 7767989 | 6877590 |
| 698956.3 | Gardnerella vaginalis | 1400E | 1715062 | 1476429 |
| 1341646.3 | Mycobacterium septicum | DSM 44393 | 6863376 | 6170700 |
| 331271.8 | Burkholderia cenocepacia | AU 1054 | 7279116 | 6257361 |
| 1198627.3 | Mycobacterium massiliense | str. GO 06 | 5068807 | 4597050 |
| 904334.4 | Staphylococcus capitis | VCU116 | 2443792 | 2093082 |
| 373665.6 | Yersinia pestis | biovar Orientalis str. | 5310846 | 4462500 |
| | | IP275 | | |
| 1176514.4 | Burkholderia glumae | AU6208 | 4833213 | 3713397 |
| 648.78 | Aeromonas caviae | strain 8LM | 4477475 | 3948033 |
| 546274.4 | Eikenella corrodens | ATCC 23834 | 2165061 | 1802454 |
| 1331258.3 | Bordetella hinzii | 8-296-03 | 9138220 | 8153910 |
| 1331253.3 | Bordetella bronchiseptica | SEAT0007 | 4046199 | 3641496 |
| 553219.3 | Campylobacter showae | RM3277 | 2060086 | 1839927 |
| 868129.3 | Prevotella bivia | DSM 20514 | 2520138 | 2157033 |
| 1463928.3 | Streptomyces sp. | NRRL WC-3683 | 11824600 | 9076380 |
| 374933.4 | Haemophilus influenzae | PittII | 1952112 | 1738566 |
| 291112.3 | Photorhabdus asymbiotica | strain ATCC 43949 | 5094138 | 4252743 |
| 562982.3 | Gemella morbillorum | M424 | 1749799 | 1493418 |
| 561522.3 | Streptococcus pyogenes | MGAS2111 | 2019649 | 1637502 |
| 546272.3 | Brucella melitensis | ATCC 23457 | 3311219 | 2892264 |
| 520999.6 | Providencia alcalifaciens | DSM 30120 | 4009093 | 3394839 |
| 1247647.3 | Bordetella holmesii | 70147 | 3766893 | 3345585 |
| 1315976.3 | Plesiomonas shigelloides | 302-73 | 3772953 | 3112590 |
| 1248902.3 | Escherichia coli | O145:H28 str. | 5737294 | 5039106 |
| | | RM13514 | | |
| 573.2239 | Klebsiella pneumoniae | strain U41 | 5857665 | 5205553 |
| 305.91 | Ralstonia solanacearum | strain 58_RSOL | 6176144 | 5524026 |
| 1208661.3 | Cronobacter dublinensis | 582 | 4699149 | 3188865 |
| 561304.4 | Mycobacterium leprae | Br4923 | 3268071 | 2219856 |
| 546275.3 | Fusobacterium periodonticum | ATCC 33693 | 2592091 | 2225847 |
| 1155096.3 | Borrelia crocidurae | str. Achema | 1526606 | 1211481 |
| 1336752.4 | Vibrio fluvialis | PG41 | 5339159 | 4544223 |
| 1841657.4 | Serratia sp. | 14-2641 | 6343511 | 5571464 |
| 883116.3 | Klebsiella oxytoca | Sep-31 | 6173601 | 5474324 |
| 29489.3 | Aeromonas enteropelogenes | strain 1999lcr | 4054080 | 2982687 |
| 314723.4 | Borrelia hermsii | DAH | 922307 | 855342 |
| 1239989.3 | Morganella morganii | SC01 | 4138684 | 3612831 |
| 452436.11 | Streptococcus dysgalactiae | subsp. equisimilis | 2217546 | 1959169 |
| | | AK5DE4288 | | |
| 1408.43 | Bacillus pumilus | B4127 | 3887138 | 3412113 |
| 418136.12 | Francisella tularensis | subsp. tularensis | 1898476 | 1690713 |
| | | WY96-3418 | | |
| 1434264.3 | Aggregatibacter | serotype e str. | 2254258 | 2001912 |
| | actinomycetemcomitans | SA2876 | | |
| 526994.3 | Bacillus cereus | AH1273 | 5790501 | 4685871 |
| 1575.5 | Leifsonia xyli | strain SE134 | 3596761 | 3319886 |
| 1496.838 | Peptoclostridium difficile | strain LIBA-5704 | 4549499 | 3829113 |
| 663.78 | Vibrio alginolyticus | strain UCD-9C | 5862215 | 5123346 |
| 997761.3 | Paenibacillus mucilaginosus | K02 | 8770140 | 7319625 |
| 575585.3 | Acinetobacter calcoaceticus | RUH2202 | 3876196 | 3252219 |
| 638315.3 | Legionella longbeachae | D-4968 | 4085043 | 3475188 |
| 1398085.3 | Inquilinus limosus | MP06 | 6934542 | 5550528 |
| 1502.206 | Clostridium perfringens | strain FORC_025 | 3343822 | 2807826 |
| 553184.4 | Atopobium rimae | ATCC 49626 | 1620446 | 1424292 |
| 498740.12 | Borrelia burgdorferi | 64b | 1485884 | 1301337 |
| 1051974.3 | Granulibacter bethesdensis | CGDNIH2 | 2736589 | 2481789 |
| 411901.7 | Bacteroides caccae | ATCC 43185 | 4563384 | 4027398 |
| 1335.2 | Streptococcus equinus | strain Sb09 | 2042259 | 1838445 |
| 306537.1 | Corynebacterium jeikeium | K411 | 2476822 | 2137170 |
| 290338.8 | Citrobacter koseri | ATCC BAA-895 | 4735357 | 4143930 |
| 693750.4 | Brucella sp. | B02 | 3296389 | 2870268 |
| 529507.6 | Proteus mirabilis | HI4320 | 4099895 | 3444813 |
| 294.17 | Pseudomonas fluorescens | strain AU20219 | 7275643 | 6473034 |
| 195.282 | Campylobacter coli | strain FB1 | 1732548 | 1621209 |
| 411555.3 | Borrelia afzelii | K78 | 1309078 | 1163688 |
| 172045.13 | Elizabethkingia miricola | strain EM_CHUV | 4286053 | 3864696 |
| 525283.3 | Fusobacterium nucleatum | subsp. nucleatum | 2221572 | 2017785 |
| | | ATCC 23726 | | |
| 553204.6 | Corynebacterium amycolatum | SK46 | 2508284 | 2162409 |
| 243160.12 | Burkholderia mallei | ATCC 23344 | 5835527 | 5014644 |
| 115711.1 | Chlamydophila pneumoniae | AR39 | 1229853 | 1109094 |
| 212042.8 | Anaplasma phagocytophilum | HZ | 1471282 | 1074840 |
| 1214102.8 | Mycobacterium fortuitum | subsp. fortuitum | 6525646 | 5833491 |
| | | DSM 46621 = ATCC | | |
| | | 6841 | | |
| 1339273.3 | Bacteroides fragilis | str. B1 (UDC16-1) | 7548423 | 6553215 |
| 211759.12 | Serratia marcescens | subsp. marcescens | 6999081 | 6083286 |
| | | strain 950165859 | | |
| 537971.5 | Helicobacter cinaedi | CCUG 18818 | 2204175 | 1958751 |
| 393117.11 | Listeria monocytogenes | FSL J1-194 | 2980528 | 2688549 |
| 243243.7 | Mycobacterium avium | 104 | 5475491 | 4913520 |
| 1513.24 | Clostridium tetani | ATCC 453 | 2890535 | 2545752 |
| 1158603.5 | Enterococcus flavescens | ATCC 49996 | 3592251 | 3123207 |
| | | [PRJNA206349] | | |
| 1328.2 | Streptococcus anginosus | strain J4211 | 1924513 | 1699176 |
| 28037.95 | Streptococcus mitis | strain SK629 | 2213700 | 1913889 |
| 592021.13 | Bacillus anthracis | str. A0248 | 5503926 | 4620222 |
| 537970.13 | Helicobacter canadensis | MIT 98-5491 | 1631445 | 1439679 |
| 596326.3 | Lactobacillus jensenii | 208-1 | 3305024 | 2933394 |
| 257311.4 | Bordetella parapertussis | 12822 | 4773551 | 4318380 |
| 766154.3 | Shigella flexneri | 1235-66 | 8597088 | 7002369 |
| 1531.8 | Clostridium clostridiiforme | strain ATCC 25537 | 5465751 | 4849840 |
| 360106.6 | Campylobacter fetus | subsp. fetus 82-40 | 1773615 | 1632693 |
| 1338011.4 | Elizabethkingia anophelis | NUHP1 | 4326189 | 3842145 |
| 537972.5 | Helicobacter pullorum | MIT 98-5489 | 1928649 | 1695156 |
| 756012.3 | Vibrio mimicus | SX-4 | 4272179 | 3752331 |
| 1405498.3 | Staphylococcus simulans | UMC-CNS-990 | 2744113 | 2361060 |
| 1161918.5 | Brachyspira pilosicoli | WesB | 2889522 | 2529369 |
| 247156.8 | Nocardia farcinica | IFM 10152 | 6292344 | 5257485 |
| 1335308.3 | Burkholderia vietnamiensis | AU4i | 9201303 | 7735050 |
| 879301.3 | Lactobacillus iners | LEAF 2053A-b | 1362693 | 1184628 |
| 1590.173 | Lactobacillus plantarum | strain 38 | 5335906 | 4397407 |
| 1121098.4 | Bacteroides massiliensis | B84634 = Timone | 4507232 | 4011354 |
| | | 84634 = DSM 17679 = | | |
| | | JCM 13223 | | |
| | | [PRJNA199226] | | |
| 592316.4 | Pantoea sp. | At-9b | 6312783 | 5446200 |
| 1162284.3 | Mycobacterium abscessus | M24 | 5486355 | 4787211 |
| 1335421.3 | Mycobacterium intracellulare | MIN_052511_1280 | 6330544 | 5657133 |
| 357244.4 | Orientia tsutsugamushi | str. Boryong | 2127051 | 1545141 |
| 1158607.4 | Enterococcus pallens | ATCC BAA-351 | 5433413 | 4743447 |
| | | [PRJNA206355] | | |
| 699034.5 | Clostridium difficile | BI1 | 4464700 | 3689148 |
| 553207.3 | Corynebacterium matruchotii | ATCC 14266 | 2835440 | 2377746 |
| 1230343.3 | Legionella anisa | str. Linanisette | 4314769 | 3752013 |
| 367737.6 | Arcobacter butzleri | RM4018 | 2341251 | 2167800 |
| 121719.1 | Pannonibacter phragmitetus | strain 31801 | 5669701 | 5012778 |
| 412419.2 | Borrelia duttonii | Ly | 1532728 | 1310154 |
| 243276.9 | Treponema pallidum | subsp. pallidum str. | 1139633 | 1063617 |
| | | Nichols | | |
| 1206782.3 | Bartonella bacilliformis | INS | 1444107 | 1189044 |
| 411465.1 | Parvimonas micra | ATCC 33270 | 1698951 | 1500612 |
| 575587.3 | Acinetobacter junii | SH205 | 3454656 | 2847876 |
| 553178.3 | Capnocytophaga gingivalis | ATCC 33624 | 2665755 | 2318955 |
| 392021.5 | Rickettsia rickettsii | str. ‘Sheila Smith’ | 1257710 | 1012374 |
| 455432.3 | Nocardia terpenica | strain IFM 0406 | 9282228 | 8331682 |
| 562981.3 | Gemella haemolysans | M341 | 2014192 | 1698903 |
| 33892.16 | Mycobacterium bovis | BCG strain 3281 | 4410431 | 4020063 |
| 350701.6 | Burkholderia dolosa | AUO158 | 6420400 | 5294946 |
| 1492.17 | Clostridium butyricum | NOR 33234 | 4922643 | 4114995 |
| 189518.3 | Leptospira interrogans | serovar Lai str. | 4691184 | 3620223 |
| | | 56601 | | |
| 412418.11 | Borrelia recurrentis | A1 | 1156178 | 1020492 |
| 1198690.3 | Brucella abortus | CNGB 759 | 3285661 | 2834922 |
| 575588.3 | Acinetobacter lwoffii | SH145 | 3462137 | 2732334 |
| 1363.19 | Lactococcus garvieae | MT14 | 2253704 | 1964214 |
| 1338.25 | Streptococcus intermedius | 567_SINT | 2069778 | 1831890 |
| 360105.8 | Campylobacter curvus | 525.92 | 1971264 | 1799760 |
| 1074000.4 | Cronobacter universalis | NCTC 9529 | 4334001 | 3838137 |
| 722438.5 | Mycoplasma pneumoniae | FH | 817207 | 753633 |
| 205920.11 | Ehrlichia chaffeensis | str. Arkansas | 1176248 | 915141 |
| 585054.5 | Escherichia fergusonii | ATCC 35469 | 4643861 | 4087158 |
| 40041.11 | Streptococcus equi | subsp. | 2149868 | 1818459 |
| | | zooepidemicus strain | | |
| | | H70 | | |
| 1208664.3 | Cronobacter sakazakii | 696 | 4872075 | 3430317 |
| 1844093.4 | Pseudomonas sp. | 22 E 5 | 14113034 | 12657564 |
| 28110.12 | Francisella philomiragia | GA01-2794 | 2152054 | 1985793 |
| 1408268.58 | Corynebacterium ulcerans | FRC58 | 2542597 | 2256624 |
| 388919.9 | Streptococcus sanguinis | SK36 | 2388435 | 2094633 |
| 1054460.4 | Streptococcus | IS7493 | 2190731 | 1889532 |
| | pseudopneumoniae | | | |
| 562973.4 | Actinomyces viscosus | C505 | 3115155 | 2599089 |
| 498743.14 | Borrelia garinii | PBr | 1263817 | 1095036 |
| 1736693.3 | Rickettsia sp. | Tenjiku01 | 1256207 | 1031916 |
| 702446.3 | Bacteroides vulgatus | PC510 | 4774434 | 4219206 |
| 1318743.3 | Candidatus Bartonella | ancashi strain 20.00 | 1467695 | 1211280 |
| 1208590.3 | Cronobacter turicensis | 564 | 4549346 | 3354072 |
| 1403335.5 | Porphyromonas gingivalis | 381 | 2378872 | 2075523 |
| 480418.6 | Mycobacterium lepromatosis | strain Mx1-22A | 3206741 | 2532285 |
| 1003202.3 | Rickettsia typhi | str. B9991CWPP | 1112957 | 837135 |

### Construction of a Sequencing Library

A further embodiment of the present invention is a method of constructing a sequencing library suitable for sequencing with any high throughput sequencing method utilizing the novel bacterial capture sequencing platform.

Accordingly, the method may include the following steps.

Nucleic acid from a sample is obtained. The sample used in the present invention may be an environmental sample, a food sample, or a biological sample. The preferred sample is a biological sample. A biological sample may be obtained from a tissue of a subject or bodily fluid from a subject including but not limited to nasopharyngeal aspirate, blood, cerebrospinal fluid, saliva, serum, urine, sputum, bronchial lavage, pericardial fluid, or peritoneal fluid, or a solid such as feces. A biological sample can also be cells, cell culture or cell culture medium. The sample may or may not comprise or contain any bacterial nucleic acids. In one embodiment, the sample is from a vertebrate subject, and in a further embodiment, the sample is from a human subject. In another embodiment, the sample comprises blood. In another preferred embodiment, the sample comprises cells, cell culture, cell culture medium or any other composition being used for developing pharmaceutical and therapeutic agents. In some embodiments, the sample is from food or a food supply.

The nucleic acids from the sample are subjected to fragmentation, to obtain a nucleic acid fragment. There are no special limitations on a type of the nucleic acid sample which may be used and there are no special limitations on means for performing the fragmentation. Any chemical or physical method which randomly fragments nucleic acid samples may be used. It is preferred that the nucleic acid sample is fragmented to obtain a nucleic acid fragment having a length of about 200 bp to about 300 bp or any other size distribution suitable for the respective sequencing platform.

After being obtained, the nucleic acid fragments can be ligated to an adaptor. In one embodiment, the adaptor is a linear adaptor. Linear adaptors can be added to the fragments by end-repairing the fragments, to obtain an end-repaired fragment; adding an adenine base to the 3′ ends of the fragment, to obtain a fragment having an adenine at the 3′ end; and ligating an adaptor to the fragment having an adenine at the 3′ end.

In some embodiments, the adaptor comprises an identifier sequence. In some embodiments, the adaptor comprises sequences for priming for amplification. In some embodiments, the adaptor comprises both an identified sequence and sequences for priming for amplification.

After the nucleic acid fragment is ligated to the adaptor, it is contacted with the oligonucleotides of the bacterial capture sequencing platform, under conditions that allow the nucleic acid fragment to hybridize to the oligonucleotides of the bacterial capture sequencing platform if the nucleic acid comprises any bacterial sequences from bacteria or genes represented in the bacterial capture sequencing platform. This step may be performed in solution or in a solid phase hybridization method, depending on the form of the bacterial capture sequencing platform.

After contact with the oligonucleotides of the bacterial capture sequencing platform, any hybridization product(s) may be subject to amplification conditions. In one embodiment, the primers for amplification are present in the adaptor ligated to the nucleic acid fragment. The resulting amplified product(s) comprise the sequencing library that is suitable to be sequenced using any HTS system now known or later developed.

Amplification may be carried out by any means known in the art, including polymerase chain reaction (PCR) and isothermal amplification. PCR is a practical system for in vitro amplification of a DNA base sequence. For example, a PCR assay may use a heat-stable polymerase and two primers: one complementary to the (+)-strand at one end of the sequence to be amplified; and the other complementary to the (−)-strand at the other end. Because the newly-synthesized DNA strands can subsequently serve as additional templates for the same primer sequences, successive rounds of primer annealing, strand elongation, and dissociation may produce rapid and highly-specific amplification of the desired sequence. PCR also may be used to detect the existence of a defined sequence in a DNA sample. In a preferred embodiment of the present invention, the hybridization products are mixed with suitable PCR reagents. A PCR reaction is then performed, to amplify the hybridization products.

In one embodiment, the sequencing library is constructed using the bacterial capture sequencing platform in a cleavable array. Nucleic acids from the sample are extracted and subjected to reverse transcriptase treatment and ligated to an adaptor comprising an identifier and sequences for priming for amplification. The oligonucleotides comprising the bacterial capture sequencing platform are synthesized using a cleavable array platform wherein the oligonucleotides are biotinylated. The biotinylated oligonucleotides are then cleaved from the solid matrix into solution with the nucleic acids from the sample to enable hybridization of the oligonucleotides comprising the bacterial capture sequencing platform to any bacterial nucleic acids in solution. After hybridization, nucleic acid(s) from the sample bound to the biotinylated oligonucleotides comprising the sequence capture platform, i.e., hybridization product(s), is collected by streptavidin magnetic beads, and amplified by PCR using the adaptor sequences as specific priming sites, resulting in an amplified product for sequencing on any known HTS systems (Ion, Illumina, 454) and any HTS system developed in the future.

In a further embodiment, the sequencing library can be directly sequenced using any method known in the art. In other words, the nucleic acids captured by the platform can be sequenced without amplification.

Methods and Systems for Simultaneous Detection, Identification, and/or Characterization of Pathogenic Bacteria and Antimicrobial Resistant Genes

The present invention includes methods and systems for the simultaneous detection of pathogenic bacteria as well as antimicrobial resistant genes or biomarkers, known or suspected to infect vertebrates, including humans, in any sample; the identification and characterization of bacteria and/or antimicrobial resistant genes or biomarkers, present in any sample; and the identification of novel bacteria and/or antimicrobial resistant genes or biomarkers in any sample, utilizing the novel bacterial capture sequencing platform.

The methods and systems of the present invention may be used to detect bacteria and/or antimicrobial resistant genes or biomarkers, known and novel, in research, clinical, environmental, and food samples. Additional applications include, without limitation, detection of infectious pathogens, the screening of blood products (e.g., screening blood products for infectious agents), biodefense, food safety, environmental contamination, forensics, and genetic-comparability studies. The present invention also provides methods and systems for detecting bacteria and/or antimicrobial resistant genes or biomarkers in cells, cell culture, cell culture medium and other compositions used for the development of pharmaceutical and therapeutic agents. Accordingly, the present invention provides methods and systems for a myriad of specific applications, including, without limitation, a method for determining the presence of bacteria and/or antimicrobial resistant genes or biomarkers in a sample, a method for screening blood products, a method for assaying a food product for contamination, a method for assaying a sample for environmental contamination, and a method for detecting genetically-modified organisms. The present invention further provides use of the system in such general applications as biodefense against bio-terrorism, forensics, and genetic-comparability studies.

The subject may be any animal, particularly a vertebrate and more particularly a mammal, including, without limitation, a cow, dog, human, monkey, mouse, pig, or rat. Preferably, the subject is a human. The subject may be known to have a pathogen infection, suspected of having a pathogen infection, or believed not to have a pathogen infection.

The systems and methods described herein support the multiplex detection of multiple bacteria and bacterial transcripts in any sample.

Thus, one embodiment of the present invention provides a system for the simultaneous detection of pathogenic bacteria known or suspected to infect vertebrates and/or antimicrobial resistant genes or biomarkers in any sample. The system includes at least one subsystem wherein the subsystem includes a bacterial capture sequencing platform as described herein. The system can also include additional subsystems for the purpose of: isolation and preparation of the nucleic acid fragments from the sample; hybridization of the nucleic acid fragments from the sample with the oligonucleotides of the bacterial capture sequencing platform to form hybridization product(s); amplification of the hybridization product(s); and sequencing the hybridization product(s).

The present invention also provides a system for the simultaneous identification and characterization of pathogenic bacteria known to infect vertebrates and/or antimicrobial resistant genes or biomarkers in any sample. The system includes at least one subsystem wherein the subsystem includes a bacterial capture sequencing platform as described herein. The system can also include additional subsystems for the purpose of: isolation and preparation of the nucleic acid fragments from the sample; hybridization of the nucleic acid fragments from the sample with the oligonucleotides of the bacterial capture sequencing platform to form hybridization product(s); amplification of the hybridization product(s); sequencing the hybridization product(s); and identification and characterization of the bacteria by the comparison between the sequences of the hybridization products and known bacteria and/or antimicrobial resistant genes or biomarkers.

In some embodiments of the foregoing systems, more than one bacteria are detected, identified, and/or characterized. In some embodiments of the foregoing systems, more than ten bacteria are detected, identified, and/or characterized. In some embodiments of the foregoing systems, more than fifty bacteria are detected, identified, and/or characterized. In some embodiments of the foregoing systems, more than one hundred bacteria are detected, identified, and/or characterized. In some embodiments of the foregoing systems, more than one hundred and fifty bacteria are detected, identified, and/or characterized. In some embodiments of the foregoing systems, more than two hundred bacteria are detected, identified, and/or characterized. In some embodiments of the foregoing systems, more than two hundred and fifty bacteria are detected, identified, and/or characterized. In some embodiments of the foregoing systems, more than three hundred bacteria detected, identified, and/or characterized. In some embodiments of the foregoing methods, all pathogenic bacteria known or suspected to infect vertebrates are detected, identified, and/or characterized. In some embodiments of the foregoing systems, some or all of the bacteria listed in Table 1 are detected, identified, and/or characterized.

The present invention also provides a system for the identification of novel bacteria and/or antimicrobial resistant genes or biomarkers in any sample. The system includes at least one subsystem wherein the subsystem includes a bacterial capture sequencing platform as described herein. The system can also include additional subsystems for the purpose of: isolation and preparation of the nucleic acid fragments from the sample; hybridization of the nucleic acid fragments from the sample with the oligonucleotides of the bacterial capture sequencing platform to form hybridization product(s); amplification of the hybridization product(s); sequencing the hybridization product(s); and identifying the bacteria and/or antimicrobial resistant genes or biomarkers as novel by the comparison between the sequences of the hybridization products and known bacteria and/or antimicrobial resistant genes or biomarkers.

Additionally, the present invention provides a method for the simultaneous detection of pathogenic bacteria known or suspected to infect vertebrates and/or antimicrobial resistant genes or biomarkers in any sample, including the steps of: obtaining the sample; isolating and preparing the nucleic acid fragments from the sample; contacting the nucleic acid fragments from the sample with the oligonucleotides of bacterial capture sequencing platform under conditions sufficient for the nucleic acid fragments and the oligonucleotides of the bacterial capture sequencing platform to hybridize; and detecting any hybridization products formed between the nucleic acid fragments and the oligonucleotides of the bacterial capture sequencing platform.

This method can also include a step to amplify and sequence the hybridization products.

The present invention provides a method for the simultaneous identification and characterization of pathogenic bacteria known or suspected to infect vertebrates and/or antimicrobial resistant genes or biomarkers in any sample, including the steps of: obtaining the sample; isolating and preparing the nucleic acid fragments from the sample; contacting the nucleic acid fragments from the sample with the oligonucleotides of the bacterial capture sequencing platform under conditions sufficient for the nucleic acid fragments and the oligonucleotides of the bacterial capture sequencing platform to hybridize; sequencing any hybridization products formed between the nucleic acid fragments and the oligonucleotides of the bacterial capture sequencing platform; comparing the sequences of the hybridization product(s) with sequences of known bacteria and/or antimicrobial resistant genes or biomarkers; and determining and characterizing the bacteria and/or antimicrobial resistant genes or biomarkers in the sample by the comparison of the sequences of the hybridization product(s) with sequences of known bacteria and/or antimicrobial resistant genes or biomarkers.

This method can also include a step to amplify the hybridization products.

In some embodiments of the foregoing methods, more than one bacteria are detected, identified, and/or characterized. In some embodiments of the foregoing methods, more than ten bacteria are detected, identified, and/or characterized. In some embodiments of the foregoing methods, more than fifty bacteria are detected, identified, and/or characterized. In some embodiments of the foregoing methods, more than one hundred bacteria are detected, identified, and/or characterized. In some embodiments of the foregoing methods, more than one hundred and fifty bacteria are detected, identified, and/or characterized. In some embodiments of the foregoing methods, more than two hundred bacteria are detected, identified, and/or characterized. In some embodiments of the foregoing methods, more than two hundred and fifty bacteria are detected, identified, and/or characterized. In some embodiments of the foregoing methods, more than three hundred bacteria detected, identified, and/or characterized. In some embodiments of the foregoing methods, all pathogenic bacteria known or suspected to infect vertebrates are detected, identified, and/or characterized. In some embodiments of the foregoing methods, some or all of the bacteria listed in Table 1 are detected, identified, and/or characterized.

The present invention provides a method for the detecting the presence of novel bacteria and/or antimicrobial resistant genes or biomarkers in any sample, including the steps of: obtaining the sample; isolating and preparing the nucleic acid fragments from the sample; contacting the nucleic acid fragments from the sample with the oligonucleotides of bacterial capture sequencing platform under conditions sufficient for the nucleic acid fragments and the oligonucleotides of the bacterial capture sequencing platform to hybridize; sequencing any hybridization products formed between the nucleic acid fragments and the bacterial capture sequencing platform; comparing the sequences of the hybridization product(s) with sequence of known bacteria and/or antimicrobial resistant genes or biomarkers; and detecting novel bacteria and/or antimicrobial resistant genes or biomarkers by the comparison of the sequences of the hybridization product(s) with sequences of known bacteria and/or antimicrobial resistant genes or biomarkers, wherein if the sequence of the hybridization product is not the same or similar enough to the known sequences, the bacteria and/or microbial resistance genes or biomarkers are novel.

This method can also include a step to amplify the hybridization products.

When practicing the methods for the determination and characterization of bacteria and/or antimicrobial resistant genes or biomarkers in a sample and methods of detecting the presence of a novel bacteria and/or antimicrobial resistant genes or biomarkers in a sample, the sequence(s) of the hybridization products are compared to the nucleic acid sequences of known bacteria and/or antimicrobial resistant genes or biomarkers. This can be done using databases in the form of a variety of media for their use.

As disclosed above, the methods of the present invention for the detection, identification and/or characterization of pathogenic bacteria known or suspected to infect vertebrates and/or antimicrobial resistant genes or biomarkers can be performed on any sample suspected of having bacteria or bacterial nucleic acids, including but not limited to biological samples, environmental samples, or food samples. A preferred sample is a biological sample. A biological sample may be obtained from a tissue of a subject or bodily fluid from a subject including but not limited to nasopharyngeal aspirate, blood, cerebrospinal fluid, saliva, serum, urine, sputum, bronchial lavage, pericardial fluid, or peritoneal fluid, or a solid such as feces. A biological sample can also be cells, cell culture or cell culture medium. The sample may or may not comprise or contain any bacterial nucleic acids.

In a preferred embodiment, the sample is from a vertebrate subject, and in a most preferred embodiment, the sample is from a human subject. In another preferred embodiment, the sample comprises cells, cell culture, cell culture medium or any other composition being used for developing pharmaceutical and therapeutic agents.

### Kits

The invention also includes reagents and kits for practicing the methods of the invention. These reagents and kits may vary.

One reagent would be the bacterial capture sequencing platform. The platform could be in the form of a collection of oligonucleotide probes which comprise sequences derived from the genome of pathogenic bacteria that are known or suspected to infect vertebrates as well as antimicrobial resistant genes. The platform could be in the form of a collection of oligonucleotide probes which comprise sequences derived from the genome of pathogenic bacteria listed in Table 1. This collection of oligonucleotide probes can be in solution or attached to a solid state. Additionally, the oligonucleotide probes can be modified for use in a reaction. A preferred modification is the addition of biotin to the probes.

The platform can also be in the form of a searchable database with information regarding the oligonucleotides including at least sequence information, length and melting temperature, and the origin.

Other reagents in the kit could include reagents for isolating and preparing nucleic acids from a sample, hybridizing the nucleic acid fragments from the sample with the oligonucleotides of the platform, amplifying the hybridization products, and obtaining sequence information.

Kits of the subject invention may include any of the above-mentioned reagents, as well as reference/control sequences that can be used to compare the test sequence information obtained, by for example, suitable computing means based upon an input of sequence information.

In addition, kits would also further include instructions.

A further embodiment is a kit for designing and/or constructing the bacterial capture sequencing platform comprising analytical tools to choose sequence information and break the coding sequences into fragments for oligonucleotides with the proper parameters for the platform including proper length, melting temperature, GC distribution, distance spaced between the oligonucleotides on the coding sequences, and percentage sequence identity. This kit could also include instructions as to database and coding sequence choice.

### EXAMPLES

### Example 1—Materials and Methods

Bacteria The following bacteria were obtained through the NIH Biodefense and Emerging Infections Research Resources Repository, NIAID, NIH: Streptococcus pneumoniae, strain SPEC6C, NR-20805; Bordetella pertussis, strain H921, NR-42457; Streptococcus agalactiae, strain SGBS001, NR-44125; Salmonella enterica subsp. enterica, strain Ty2 (Serovar Typhi), NR-514; Neisseria meningitidis, strain 98008, NR-30536; Klebsiella pneumoniae, isolate 1, NR-15410; Escherichia coli, strain B171, NR-9296; Vibrio cholerae, strain 395, NR-9906; and Campylobacter jejuni, strain HB95-29, NR-402. Staphylococcus aureus ATCC®25923 and ATCC®29213 were acquired from American Type Culture Collection. Bacterial nucleic acids were extracted using Allprep mini DNA/RNA kit (Qiagen, Hilden, Germany).
Nucleic acid extraction Total nucleic acid from bacterial cells, whole blood spiked with bacteria or bacterial nucleic acids were extracted using Allprep mini DNA/RNA kit (Qiagen, Hilden, Germany) and quantitated by NanoDrop One (Wilmington, Del., USA) or Bioanalyzer 2100 (Agilent, Santa Clara, Calif., USA). Bacterial nucleic acid (NA) and genome equivalents were quantitated by agent-specific quantitative TaqMan real-time PCR.
Agent-specific quantitative TaqMan real-time PCR and standards Primers and probes for quantitative PCR (qPCR) were selected in conserved single-copy genes of the investigated bacterial species with Geneious v10.2.3) (Table 2). Standards for quantitation were generated by cloning a fragment of the targeted gene spanning the primers into pGEM-T Easy vector (Promega, Madison, Wis., USA). Recombinant plasmid DNA was purified using Mini Plasmid Prep Kit (Qiagen). Linearized plasmid DNA concentration was determined using NanoDrop One, and copy numbers adjusted by dilution in Tris-HCl, pH 8 with 1 ng/ml salmon sperm DNA.

TABLE 2
Primers and Probes used for qPCR

| | Gene | | |
|---|---|---|---|
| Bacteria | Target | Primers | Accession # |
| M. tuberculosis | pncA | pnc270F TCTCGGCCAGGATGAATTTG | NC_000962 |
| | | (SEQ ID NO: 1) | |
| | | pnc340P TTTGAAGGTGGGGCGCACGA | |
| | | (SEQ ID NO: 2) | |
| | | pnc429R CGCTACCACCATTTCTTCGA | |
| | | (SEQ ID NO: 3) | |
| K. pneumoniae | hyn | hln240F AAACGGCTATCTCTGGAAGC | NC_016845 |
| | | (SEQ ID NO: 4) | |
| | | h1n335P CCCACCACCAGCAGACGAACTT | |
| | | (SEQ ID NO: 5) | |
| | | h1n376R TGTACTTCTTGTTGGCCTCG | |
| | | (SEQ ID NO: 6) | |
| E. coli | eaeA | int2253F TGCCCCGTTGAGTATTGATG | FM180568 |
| | | (SEQ ID NO: 7) | |
| | | int2292P AGCCCCCGTGATACCAGTACCA | |
| | | (SEQ ID NO: 8) | |
| | | int2357R GCCTGTAGCTTAACCTGACC | |
| | | (SEQ ID NO: 9) | |
| S. pneumoniae | pln | pln186F AACAGCTACCAACGACAGTC | NC_003098 |
| | | (SEQ ID NO: 10) | |
| | | pln213P TCCACTACGAGAAGTGCTCCAGGA | |
| | | (SEQ ID NO: 11) | |
| | | pln279R ATCAACCGCAAGAAGAGTGG | |
| | | (SEQ ID NO: 12) | |
| C. jejuni | hipO | hip57F ATAGGAAAAACAGGCGTTGT | NC_002163 |
| | | (SEQ ID NO: 13) | |
| | | hip119P AGGCAAAGCATCCATATCTGCACGA | |
| | | (SEQ ID NO: 14) | |
| | | hip206R ACCACAAGCATGCATTACAT | |
| | | (SEQ ID NO: 15) | |
| N. meningitidis | ctrA | ctr935F CGGCAGAACGTCAGGATAAA | NC_003112 |
| | | (SEQ ID NO: 16) | |
| | | ctr973P GGCAGTGAGGCAGAGATTCCA | |
| | | (SEQ ID NO: 17) | |
| | | ctr1026R ATGCGCATCAGCCATATTCA | |
| | | (SEQ ID NO: 18) | |
| B. pertussis | ptxA | ptx136F TGCGTTTTGATGGTGCCTAT | AXSM02000007 |
| | | (SEQ ID NO: 19) | |
| | | ptx205P CGGTACCATCGCGCGACTTT | |
| | | (SEQ ID NO: 20) | |
| | | ptx257R CAATCCAACACGGCATGAAC | |
| | | (SEQ ID NO: 21) | |
| V. cholerae | gbpA | gbp594R GTCGATCACGTTGTAGAAGG | NC_012583 |
| | | (SEQ ID NO: 22) | |
| | | gbp512P TGCCTGAGCGCGAAGGGTAT | |
| | | (SEQ ID NO: 23) | |
| | | gbp450F GTTCTGTGTCGTTGAAGGAA | |
| | | (SEQ ID NO: 24) | |
| S. typhi | staG | STPr CATTTGTTCTGGAGCAGGCTGACGG | AE014613 |
| (source- Nga et | | (SEQ ID NO: 25) | |
| al. 2010) | | ST-Frt CGCGAAGTCAGAGTCGACATAG | |
| | | (SEQ ID NO: 26) | |
| | | ST-Rrt AAGACCTCAACGCCGATCAC | |
| | | (SEQ ID NO: 27) | |
| S. agalactiae | cpsB | cps536F GCTTTAAGAAAAGAGCCCGT | CP019978 |
| | | (SEQ ID NO: 28) | |
| | | cps576P TGCATATCACTCGCTACAAAATGCACT | |
| | | (SEQ ID NO: 29) | |
| | | cps637R CTTCTGCTAAAAATGGCGGT | |
| | | (SEQ ID NO: 30) | |

Probe design The objective was to target all known human bacterial pathogens as well as any known antimicrobial resistant genes and virulence factors. Known human pathogenic bacteria were selected from the available bacterial genomes in the PATRIC database (Wattam et al. 2017). Included were all species for which at least one strain or isolate is annotated as “human-related” and “pathogenic. One genome was selected per species due to probe number limitations. Other bacterial species that were considered to have high potential to become pathogenic were added. The final list contained 307 species (Table 1), including all 19 bacterial species listed in the priority list from of the Child Health and Mortality Prevention program of the Bill and Melinda Gates Foundation.

The protein coding sequences from the selected genomes of the 307 species were extracted and combined with the full dataset of 2,169 antimicrobial resistant gene sequences in the CARD database (Jia et al. 2017) and the 30,178 virulence factor genes in the VFDB database (Chen et al. 2016; Chen et al. 2004). The combined target sequence dataset was clustered at 96% sequence identity (resulting in 1,007,426 genes) and sent to the bioinformatics core of Roche-NimbleGen (Madison, Wis., USA), where sequences were subjected to further filtration based on printing considerations. Probe lengths were refined by adjusting their start/stop positions to constrain the melting temperature. The final library comprised 4,220,566 oligonucleotides averaging 75 nt in length. The average interprobe distance between the probes along the targeted bacterial proteome, virulence, and AMR targets was 121 nucleotides.

Unbiased high-throughput sequencing (UHTS) Double-stranded cDNA was sheared to an average fragment size of 200 bp (E210 focused ultrasonicator; Covaris, Woburn, Mass., USA). Sheared products were purified using AxyPrep Mag PCR cleanup beads (Axygen/Corning, Corning, N.Y., USA), and libraries constructed using KAPA library preparation kits (Wilmington, Mass., USA) with input quantities of 10-100 ng DNA. Libraries were purified (AxyPrep) and quantitated by Bioanalyzer (Agilent) prior to sequencing on an Illumina MiSeq platform v3 (San Diego, Calif., USA).
Bacterial capture sequencing (BacCapSeq) Nucleic acid preparation, shearing and library construction was the same as for unbiased HTS, except for the use of Roche/NimbleGen SeqCap EZ indexed adapter kits. The quality and quantity of libraries were checked using a Bioanalyzer (Agilent). Libraries were mixed with a SeqCap HE universal oligonucleotide, SeqCap HE index blocking oligonucleotides, and COT DNA and vacuum evaporated at 60° C. Dried samples were mixed with hybridization buffer and hybridization component A (Roche-NimbleGen) prior to denaturation at 95° C. for 10 minutes. The BacCap probe library was added and hybridized at 47° C. for 12 hours in a standard PCR thermocycler. SeqCap Pure capture beads (Roche-NimbleGen) were washed twice, mixed with the hybridization mix, and kept at 47° C. for 45 minutes with vortexing for 10 seconds every 10 to 15 minutes. The streptavidin capture beads complexed with biotinylated BacCapSeq probes were trapped (DynaMag-2 magnet; Thermo, Fisher) and washed once at 47° C. and then twice more at room temperature with wash buffers of increasing stringency. Finally, beads were suspended in 50 ul water and directly subjected to posthybridization PCR (SeqCap EZ accessory kit V2; Roche-NimbleGen). The PCR products were purified (Agencourt Ampure DNA purification beads; Beckman Coulter, Brea, Calif., USA) prior to sequencing on an Illumina MiSeq platform v3. The time required for extraction, library construction, hybridization, generation of 150 bp single reads, and bioinformatic analysis was approximately 70 hours.
Data analysis and bioinformatics pipeline Each individual sample yielded an average of 5 million 100-bp single-end reads. The demultiplexed FastQ files were adapter trimmed using Cutadapt v1.13 (Martin 2011). Adapter trimming was followed by generation of quality reports using FastQC v0.11.5 and filtering with PRINSEQ v 0.20.3 (Schieder and Edwards 2011). Host background levels were determined by mapping the filtered reads against the human genome using Bowtie2 v2.0.6 (Langmead and Salzberg 2012). The host-subtracted reads were de-novo assembled using Megahit v1.0.4-beta (Li et al. 2015), contigs and unique singletons were subjected to homology search using MegaBlast against the GenBank nucleotide database (Clark et al. 2016). The genomes of the tested bacteria were mapped with Bowtie2 against the filtered dataset to visualize the depth and the genome recovery in IGV (Robinson et al. 2011; Thorvaldsdottir et al. 2013). Targets with read counts above a 0.001% cut-off (>10 reads/1 million quality and host filtered reads) were rated positive.

For transcriptional analyses, MiSeq reads were aligned using the STAR read mapping package (Dobin et al. 2013). Expression data were extracted from each sample using featureCounts (Liao et al. 2014), and the results were compiled into a master data file representing transcript counts for each gene. These data were normalized based on the number of reads sequenced for each sample, and the data were sorted by strain (AMR+/AMR−), time point, and antibiotic treatment to identify genes with differences in growth patterns based on these metrics.

### Example 2—Probe Design Strategy

A probe set comprising of 4.2 million oligonucleotides was assembled based on the Pathosystems Resource Integration Center (PATRIC) database (Wattam et al. 2017), representing 307 bacterial species that included all known human pathogenic species. The probe set also represented all known antimicrobial resistant genes and virulence factors based on sequences in the Comprehensive Antibiotic Resistance Database (CARD) (Jia et al. 2016) and Virulence Factor Database (VFDB) (Chen et al. 2016; Chen et al. 2004).

Probes were selected along the coding sequences of the 307 targeted bacteria (see Table 1) with an average length of 75 nucleotides (nt) to maintain a probe melting temperature (Tm) with a mean of 79° C. The average interval between probes along annotated protein coding sequences targeted for capture was 121 nt. The probes capture fragments that include sequences contiguous to their targets, thus, near complete protein coding sequences were recovered.

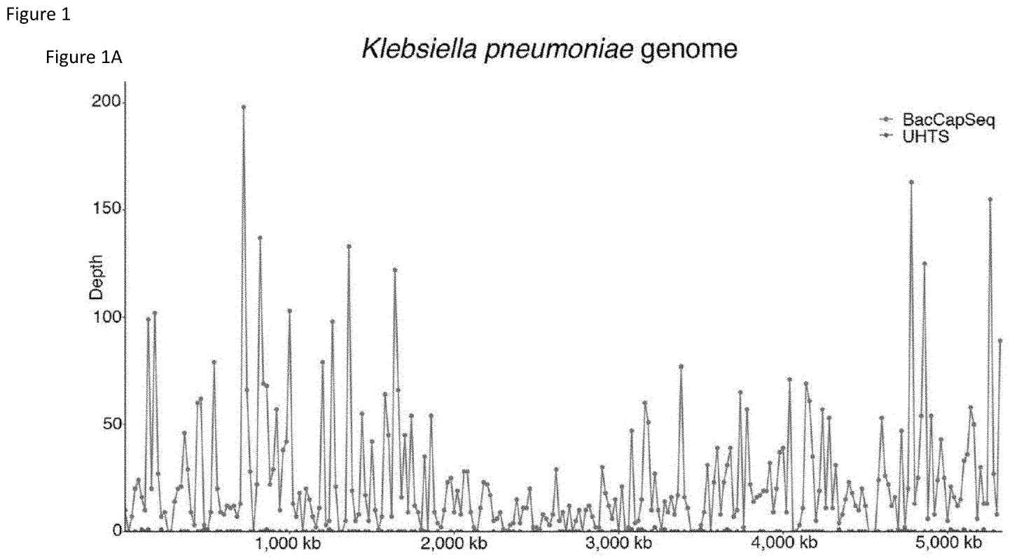

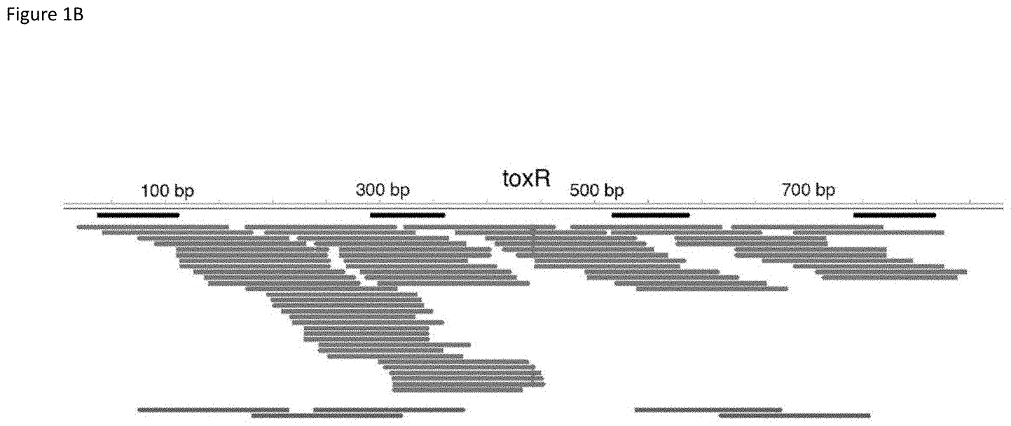

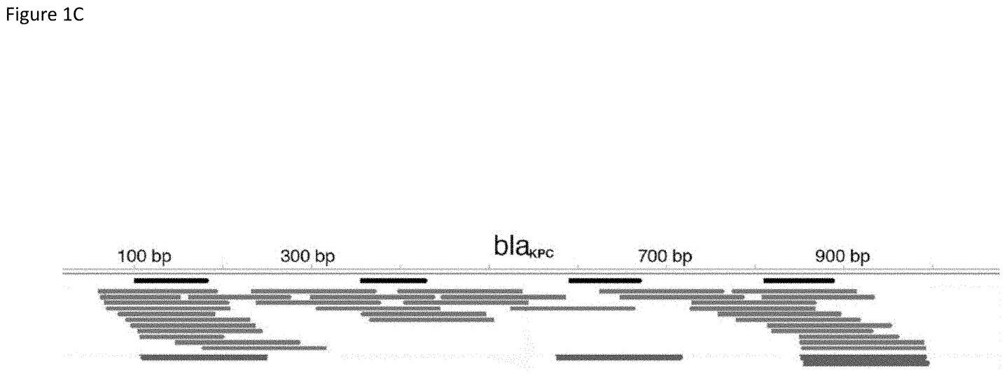

An example with Klebsiella pneumoniae is shown in FIG. 1A. Probes based on the CARD and VFDB databases ensured coverage of AMR genes and virulence factors, as illustrated by detection of the toxR virulence factor regulator in Vibrio cholerae (FIG. 1B) and bla_{KPC} AMR gene in K. pneumoniae (FIG. 1C).

### Example 3—Assessment of BacCapSeq Performance Using Whole Blood Spiked with Bacterial Nucleic Acid

The efficiency of BacCapSeq versus conventional unbiased high throughput sequencing (UHTS) was assessed in side-by-side comparisons of data obtained with five million reads per sample. First extracts of whole blood spiked with DNA from Bordetella pertussiss (B. pertussis), Escherichia coli (E. coli), Neisseria meningitidis (N. meningitidis), Salmonella enterica serovar Typhi (S. enterica), Streptococcus agalactiae (S. agalactiae), Streptococcus pneumoniae (S. pneumoniae), Vibrio cholerae (V. cholerae) and Campylobacter jejuni (C. jeuni) at concentrations ranging from 40 to 40,000 copies per milliliter were assessed. BacCapSeq yielded up to 100-fold more reads and higher genome coverage for all bacterial targets tested when compared to UHTS (Table 3). The enhanced performance of BacCapSeq was particularly pronounced at lower copy concentrations.

TABLE 3
Read Counts and Genome Coverage in Whole Blood Extracts spiked with Bacterial
DNA using BacCapSeq and UHTS

| | | | | Bacterial | Bacterial | | Genome | Genome |
|---|---|---|---|---|---|---|---|---|
| | Genome | Coding | Load | Read | Read | | Coverage | Coverage |
| | length | regions | (copies/ | count ^{a} | count ^{a} | Fold | (%) | (%) |
| Species | (nt) | (%) | ml) | BacCapSeq | UHTS | increase | BacCapSeq | UHTS |
| B. pertussis | 4,386,396 | 89 | 40,000 | 329,926 | 203563 | 2 | 100 | 99 |
| | | | 4,000 | 295,830 | 19,362 | 15 | 98 | 93 |
| | | | 400 | 155,109 | 2,189 | 71 | 73 | 29 |
| | | | 40 | 8,596 | 191 | 45 | 9 | 3 |
| E. coli | 4,965,553 | 88 | 40,000 | 281,925 | 77,793 | 4 | 82 | 81 |
| | | | 4,000 | 253,423 | 7,558 | 34 | 81 | 60 |
| | | | 400 | 132,168 | 848 | 156 | 64 | 11 |
| | | | 40 | 8,614 | 70 | 123 | 8 | 1 |
| N. Meningitidis | 2,272,360 | 86 | 40,000 | 228,937 | 72,532 | 3 | 93 | 93 |
| | | | 4,000 | 206,096 | 6,995 | 29 | 91 | 82 |
| | | | 400 | 109,446 | 824 | 133 | 79 | 22 |
| | | | 40 | 6,609 | 68 | 97 | 13 | 2 |
| S. enterica | 4,791,961 | 88 | 40,000 | 25,155 | 8,620 | 3 | 94 | 63 |
| | | | 4,000 | 22,726 | 841 | 27 | 68 | 12 |
| | | | 400 | 12,009 | 102 | 118 | 16 | 1 |
| | | | 40 | 796 | 10 | 80 | 1 | 0 |
| S. agalactiae | 2,198,785 | 89 | 40,000 | 8,467 | 4,701 | 2 | 85 | 67 |
| | | | 4,000 | 7,905 | 473 | 17 | 63 | 15 |
| | | | 400 | 4,206 | 58 | 73 | 13 | 2 |
| | | | 40 | 298 | 4 | 75 | 1 | 0 |
| S. pneumoniae | 2,038,615 | 86 | 40,000 | 8,419 | 2,290 | 3 | 91 | 56 |
| | | | 4,000 | 7,795 | 280 | 28 | 66 | 10 |
| | | | 400 | 4,124 | 30 | 137 | 14 | 1 |
| | | | 40 | 275 | 2 | 138 | 1 | 0 |
| V. cholerae | 6,048,147 | 87 | 40,000 | 11,291 | 5,381 | 2 | 97 | 64 |
| | | | 4,000 | 10,124 | 530 | 19 | 66 | 12 |
| | | | 400 | 5,127 | 61 | 84 | 12 | 1 |
| | | | 40 | 315 | 6 | 53 | 1 | 0 |
| C. jejuni | 1,641,481 | 94 | 40,000 | 5,904 | 4,195 | 1 | 89 | 73 |
| | | | 4,000 | 5,460 | 415 | 13 | 63 | 17 |
| | | | 400 | 3,223 | 52 | 62 | 14 | 2 |
| | | | 40 | 235 | 3 | 78 | 1 | 0 |

^{a} Bacterial reads per 1 million reads are shown without applying a cutoff threshold.

### Example 4—Assessment of BacCapSeq Performance Using Whole Blood Spiked with Bacterial Cells

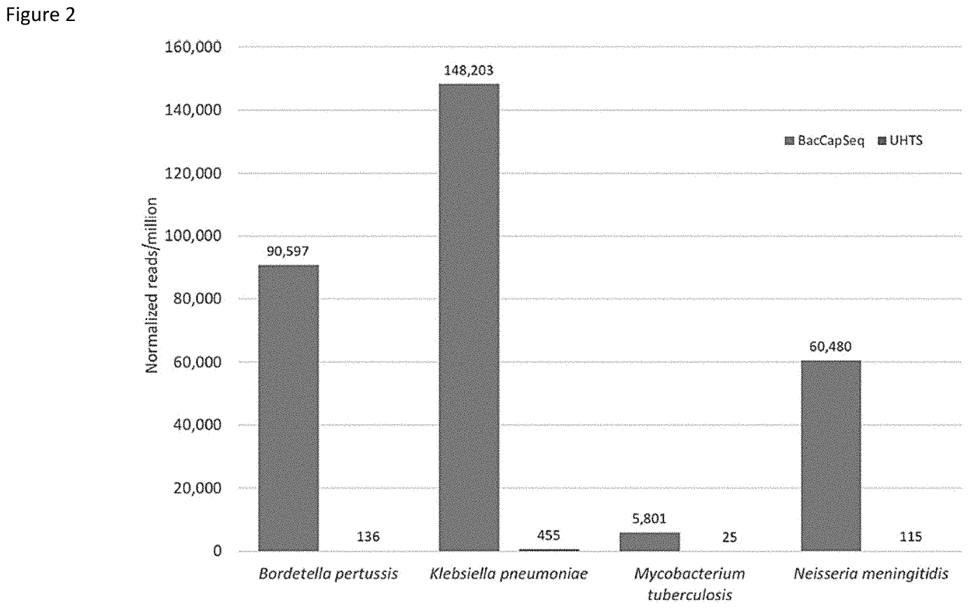

Performance was tested with whole blood spiked with Klebsiella pneumoniae (K. pneumoniae), B. pertussis, N. meningitidis, S. pneumoniae and Mycobacterium tuberculosis (M. tuberculosis) bacterial cells. Nucleic acid was extracted from spiked samples and processed for BacCapSeq or UHTS. Similar to Example 3, BacCapSeq yielded more reads and higher genome coverage than unbiased HTS, with up to 1,500-fold increased read counts (Table 4 and FIG. 2).

TABLE 4
Read Counts and Genome Coverage in Whole Blood Extracts spiked with Bacterial
Cells using BacCapSeq and UHTS

| | | | | Bacterial | Bacterial | | Genome | Genome |
|---|---|---|---|---|---|---|---|---|
| | Genome | Coding | Load | Read | Read | | Coverage | Coverage |
| | length | regions | (copies/ | count ^{a} | count ^{a} | Fold | (%) | (%) |
| Species | (nt) | (%) | ml) | BacCapSeq | UHTS | increase | BacCapSeq | UHTS |
| B. pertussis | 4,386,396 | 89 | 40,000 | 90,597 | 136 | 694 | 82 | 9 |
| | | | 4,000 | 14,858 | 16 | 979 | 39 | 5 |
| | | | 400 | 1,622 | 2 | 725 | 13 | 1 |
| | | | 40 | 296 | 1 | 508 | 8 | 0 |
| K. pneumoniae | 5,333,942 | 89 | 40,000 | 148,203 | 455 | 339 | 92 | 6 |
| | | | 4,000 | 16,929 | 40 | 442 | 58 | 1 |
| | | | 400 | 2,771 | 5 | 551 | 18 | 0 |
| | | | 40 | 522 | 0 | NA^{b} | 5 | 0 |
| M. tuberculosis | 4,411,532 | 91 | 40,000 | 5,801 | 25 | 243 | 46 | 0 |
| | | | 4,000 | 845 | 3 | 287 | 9 | 0 |
| | | | 400 | 14 | 0 | NA | 0 | 0 |
| | | | 40 | 6 | 0 | NA | 0 | 0 |
| N. meningitidis | 2,272,360 | 86 | 40,000 | 60,480 | 115 | 546 | 90 | 6 |
| | | | 4,000 | 6,894 | 8 | 908 | 57 | 0 |
| | | | 400 | 1,454 | 1 | 1,562 | 23 | 0 |
| | | | 40 | 151 | 0 | NA | 6 | 0 |
| S. pneumoniae | 2,038,615 | 86 | 40,000 | 3,070 | 6 | 506 | 43 | 0 |
| | | | 4,000 | 588 | 1 | 948 | 13 | 0 |
| | | | 400 | 35 | 0 | NA | 1 | 0 |
| | | | 40 | 4 | 0 | NA | 0 | 0 |

^{a} Bacterial reads per 1 million reads are shown without applying a cutoff threshold.
^{b}NA not applicable because fold increase was not calculated for results with less than 1 read.

### Example 5—Assessment of BacCapSeq Performance Using Clinical Cultured Blood Samples

The utility of BacCapSeq was tested in analysis of blood culture samples obtained from the Clinical Microbiology Laboratory at NewYork-Presbyterian Hospital/Columbia University Medical Center. Patient blood was collected into conventional BacTec blood culture flasks and incubated until flagged growth-positive by the BD BacTec Automated Blood Culture System (Becton Dickinson). The use of BacCapSeq recovered near full genome sequences and identified antimicrobial resistant genes that matched standard microbiology laboratory antimicrobial sensitivity testing (AST) profiles (Tables 5 and 6).

TABLE 5
Detection of Pathogenic Bacteria and Antimicrobial Resistant Genes in Cultured Blood Samples

| | | Total no. | | | | |
|---|---|---|---|---|---|---|
| | | of | | Genome | | |
| | No. of | mapped | Bacterium | Coverage | AST | Significant AMR |
| Sample | raw reads | reads | identified | (%) | profile^{a} | gene(s) detected |
| 1 | 2,833,697 | 2,709,612 | Pseudomonas | 87 | TET (R), | mexA to —N, —P, —Q, —S, |
| | | | aeruginosa | | MERO (I) | —V, and —W combined |
| | | | | | | with oprM |
| 2 | 8,322,222 | 7,126,518 | Escherichia | 81 | AMP (I), | TEMS |
| | | | coli | | CEF (I) | (115, 4, 80, 6, 153, 143, 79) |
| | | | | | | combined with |
| | | | | | | numerous efflux pump |
| | | | | | | antiporters (including |
| | | | | | | most prominently acrF, |
| | | | | | | cpxR, or H-NS) |
| 3 | 5,768,129 | 5.,96,360 | Morganella | 90 | AMP (R), | Numerous DHA |
| | | | morganii | | CEPH (R), | complex β-lactamases |
| | | | | | AZT (I) | (DBA−20, −17, −21, −1, −19), |
| | | | | | | combined with |
| | | | | | | efflux pump antiporters |
| | | | | | | acrB and smeB; cpxR, |
| | | | | | | related to aztreonam |
| | | | | | | resistance |
| 4 | 5,749,637 | 4,774,301 | Haemophilus | 92 | NA | hmrM |
| | | | influenzae | | | |

^{a}antimicrobial sensitivity test (AST) profile: AMP, ampicillin; AZT, aztreonam; CEF, cefoxitin; CEPH, cefazolin/ceftazidime/ceftriaxone; MERO, meropenem; TET, tetracycline. R, resistant; I, intermediate rating; NA, not applicable.

TABLE 6
Antimicrobial Resistant Genes Detected in Cultured Blood Samples

| | |
|---|---|
| Reads^{a} | AMR Gene |

Sample 1, Pseudomonas aeruginosa (Bacterium Identified)

| | |
|---|---|
| 5654 | mexB |
| 4268 | mexD |
| 3925 | mexF |
| 2257 | mexI |
| 2121 | TriC |
| 2016 | mexK |
| 1995 | mexW |
| 1942 | mexQ |
| 1206 | amrB |
| 1200 | arnA |
| 1156 | mexA |
| 1093 | mexN |
| 848 | oprM |
| 791 | PmrB |
| 740 | mexS |
| 698 | oprJ |
| 692 | OXA-50 |
| 688 | OpmH |
| 564 | opmD |
| 535 | PDC-7 |
| 504 | mexP |
| 500 | nfxB |
| 490 | catB7 |
| 470 | mexE |
| 456 | opmE |
| 442 | mexH |
| 424 | mexV |
| 359 | mexJ |
| 358 | mexC |
| 352 | TriA |
| 336 | TriB |
| 329 | mexL |
| 320 | mexM |
| 250 | APH(3′)-IIb |
| 233 | nalD |
| 230 | oprN |
| 219 | emrE |
| 210 | mexG |
| 208 | PDC-5 |
| 113 | amrA |
| 107 | FosA |
| 99 | mexX |
| 55 | mdtP |
| 47 | mexD |

Sample 2, Escherichia coli (Bacterium Identified)

| | |
|---|---|
| 2787 | emrR |
| 2730 | adiY |
| 2632 | emrA |
| 2610 | mdfA |
| 2521 | leuO |
| 2226 | PmrC |
| 2201 | mdtE |
| 2089 | baeS |
| 2003 | gadW |
| 1869 | PmrB |
| 1846 | TEM-115 |
| 1784 | mdtN |
| 1696 | sat-1 |
| 1668 | baeR |
| 1546 | mdtP |
| 1462 | emrK |
| 1447 | acrE |
| 1442 | dfrA1 |
| 1410 | H-NS |
| 1386 | TEM-4 |
| 1370 | gadE |
| 1361 | aadA24 |
| 1239 | kdpE |
| 1236 | acrB |
| 1185 | aminocoumarin |
| 1147 | dfrA1 |
| 1035 | acrS |
| 939 | marA |
| 896 | TEM-80 |
| 869 | acrA |
| 608 | emrE |
| 590 | gadX |
| 571 | evgA |
| 525 | aadA8 |
| 471 | aadA |
| 364 | TEM-6 |
| 152 | TEM-153 |
| 135 | TEM-143 |
| 132 | TEM-79 |
| 124 | aadA6 |
| 118 | ACT-24 |
| 97 | MIR-2 |
| 94 | mdtK |

Sample 3, Morganella morganii (Bacterium Identified)

| | |
|---|---|
| 2482 | DHA-20 |
| 1176 | DHA-17 |
| 1172 | DHA-21 |
| 868 | acrB |
| 775 | DHA-1 |
| 701 | smeB |
| 599 | CRP |
| 433 | acrD |
| 321 | DHA-19 |
| 197 | catII |
| 188 | YojI |
| 164 | cpxR |
| 143 | mfd |
| 77 | mdtF |

Sample 4, Haemophilus influenzae (Bacterium Identified)

| Reads | AMR Gene |
|---|---|
| 8761 | hmrM |

^{a}Only read counts above the positivity threshold of <10/million reads are shown.

### Example 6—BacCapSeq Performance with Human Blood Samples

Blood samples from two immunosuppressed individuals with HIV/AIDS and sepsis of unknown cause were extracted and processed for BacCapSeq and UHTS analysis in parallel. A causative agent was identified by both methods, however, BacCapSeq yielded higher numbers of relevant reads and better genome coverage (FIG. 3). Salmonella enterica was detected in one patient. The other patient had evidence of coinfection with both S. pneumoniae and Gardnerella vaginalis.

### Example 7—BacCapSeq-Facilitated Discovery of Expressed AMR Genes

The current probe set specifically captured all AMR genes present in the CARD database. Demonstrating the presence of an AMR gene is not equivalent to finding evidence for its functional expression. To address this challenge, BacCapSeq was used to pursue biomarkers in bacteria exposed to antibiotics. Ampicillin-sensitive and -resistant strains of Staphylococcus aureus at an inoculum of 1000 CFU/ml were cultured in the presence or absence of antibiotic for 45, 90, and 270 minutes. RNA was then extracted for BacCapSeq and UHTS to perform transcriptomic analysis to find biomarkers that differentiated ampicillin-sensitive and ampicillin-resistant S. aureus.

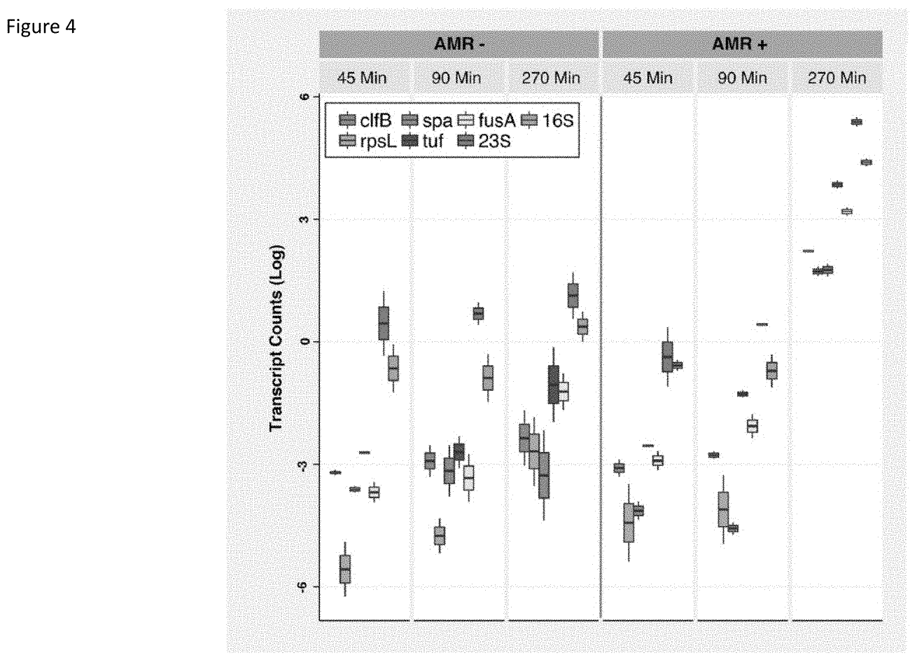

BacCapSeq, but not UHTS, enabled the discovery of transcripts that were differentially expressed between 90 minute and 270 minutes of antibiotic exposure (FIG. 4). These biomarkers included constitutive genes that reflect bacterial replication but also strain- and species-specific markers such as 16S and 23S RNA, elongation factors TU (tuf) and G (fusA), protein A (spa), clumping factor B (clfB), or ribosomal protein S12 (rpsL).

### REFERENCES

Bourbeau et al. 2005. Routine incubation of BacT/ALERT FA and FN blood culture bottles for more than 3 days may not be necessary. J Clin Microbiol 43:2506-2509.
Chen et al. 2016. VFDB 2016: hierarchical and refined dataset for big data analysis—10 years on. Nucleic Acids Res 44:D694-D697.
Chen et al. 2004. VFDB: a reference database for bacterial virulence factors. Nucleic Acids Res 33:D325-D328.
Clark et al. 2016. GenBank. Nucleic Acids Res 44:D67-D72. 34.
CLSI. 2007. Principles and procedures for blood cultures; approved guideline. CLSI document M47-A. Clinical and Laboratory Standards Institute, Wayne, Pa.
Cockerill et al. 2004. Optimal testing parameters for blood cultures. Clin Infect Dis 38:1724-1730.
Dobin et al. 2013. STAR: ultrafast universal RNA-seq aligner. Bioinformatics 29:15-21.
Golkar et al. 2014. Bacteriophage therapy: a potential solution for the antibiotic resistance crisis. J Infect Dev Ctries 8:129-136.
Howell and Davis. 2017. Management of sepsis and septic shock. JAMA 317:847-848.
Jia et al. 2016. CARD 2017: expansion and model-centric curation of the comprehensive antibiotic resistance database. Nucleic Acids Res 45:D566-D573.
Langmead and Salzberg 2012. Fast gapped-read alignment with Bowtie 2. Nat Methods 9:357.
Lee et al. 2007. Detection of bloodstream infections in adults: how many blood cultures are needed? J Clin Microbiol 45:3546-3548.
Li et al. 2015. MEGAHIT: an ultra-fast single-node solution for large and complex metagenomics assembly via succinct de Bruijn graph. Bioinformatics 31:1674-1676.
Liao et al. 2014. featureCounts: an efficient general purpose program for assigning sequence reads to genomic features. Bioinformatics 30:923-930.
MacVane and Nolte. 2016. Benefits of adding a rapid PCR-based blood culture identification panel to an established antimicrobial stewardship program. J Clin Microbiol 54:2455-2463.
Martin 2011. Cutadapt removes adapter sequences from highthroughput sequencing reads. EMBnet J 17:10-12.
Rhee et al. 2017. Incidence and trends of sepsis in US hospitals using clinical vs claims data, 2009-2014. JAMA 318:1241-1249.
Robinson et al. 2011. Integrative genomics viewer. Nat Biotechnol 29:24.
Schmieder and Edwards 2011. Quality control and preprocessing of metagenomic datasets. Bioinformatics 27:863-864.
Thorvaldsdóttir et al. 2013. Integrative Genomics Viewer (IGV): high-performance genomics data visualization and exploration. Brief Bioinform 14:178-192.
Wattam et al. 2017. Improvements to PATRIC, the all-bacterial bioinformatics database and analysis resource center. Nucleic Acids Res 45:D535-D542.

1. A computer program product stored on a memory device adapted to cause a computer to carry out a method of designing and/or constructing a bacterial capture sequencing platform comprising oligonucleotides for the simultaneous detection, identification and/or characterization of pathogenic bacteria known or suspected to infect vertebrates and antimicrobial resistant genes or biomarkers, comprising:
a. obtaining nucleotide sequences of the genomes of at least one bacteria listed in Table 1;
b. extracting and pooling coding sequences from the nucleotide sequences obtained from the genomes of at least one bacteria listed in Table 1;
c. breaking the coding sequences into fragments, wherein the fragments are about 50 to about 100 nucleotides in length and are tiled across the coding sequences at specific intervals to obtain sequence information to design oligonucleotides that selectively hybridize to genomes of pathogenic bacteria; and
d. outputting the bacterial capture sequencing platform comprising oligonucleotides with sequence information, length, melting temperature, and bacterial origin of each oligonucleotide for which sequence information was obtained.

2. The method of claim 9, further comprising obtaining the nucleotide sequences of all of the known antimicrobial resistant genes from the Comprehensive Antibiotic Resistance Database (CARD) and extracting and pooling coding sequences from the nucleotide sequences obtained from CARD with the nucleotide sequences from the genomes of the at least one bacteria.

3. The method of claim 2, further comprising obtaining the nucleotide sequences of all of the virulence factors from the Virulence Factor Database (VFDB) and extracting and pooling the coding sequences obtained from VFDB with the known antimicrobial resistant genes from the Comprehensive Antibiotic Resistance Database (CARD) and the nucleotide sequences from the genomes of the at least one bacteria.

4. The method of claim 9, wherein the length of the fragments is adjusted such that the melting temperatures of all of the fragments are in a range of about 62° C. to about 101° C.

5. The method of claim 9, wherein the length of the fragments is adjusted such that the melting temperatures of all of the fragments are about 82.7° C.

6. The method of claim 9, wherein length of the fragments is about 75 nucleotides.

7. (canceled)

8. (canceled)

9. A method of designing and/or constructing a bacterial capture sequencing platform comprising oligonucleotides for the simultaneous detection, identification and/or characterization of pathogenic bacteria known or suspected to infect vertebrates and antimicrobial resistant genes or biomarkers, comprising:
a. obtaining nucleotide sequences of the genomes of at least one bacteria listed in Table 1;
b. extracting and pooling coding sequences the nucleotide sequences obtained from the genomes of at least one bacteria listed in Table 1;
c. breaking the coding sequences into fragments, wherein the fragments are about 50 to about 100 nucleotides in length and are tiled across the coding sequences at specific intervals to obtain sequence information to design oligonucleotides that selectively hybridize to genomes of pathogenic bacteria; and
d. synthesizing the oligonucleotides for which the sequence information was obtained.

10. The method of claim 9, wherein the oligonucleotides are chosen from the group consisting of DNA, RNA, Bridged Nucleic Acids, Locked Nucleic Acids, and Peptide Nucleic Acids.

11. The method of claim 9, wherein the oligonucleotides are synthesized on a cleavable microarray.

12. The method of claim 9, wherein the oligonucleotides are modified to comprise a composition for binding to a solid support, chosen from the group consisting of biotin, digoxygenin, ligands, small organic molecules, small inorganic molecules, apatamers, antigens, antibodies, and substrates.

13. (canceled)

14. A bacterial capture sequencing platform for the simultaneous detection, identification and/or characterization of pathogenic bacteria known or suspected to infect vertebrates, and/or antimicrobial resistant genes or biomarkers, constructed by the computer program product of claim 1, wherein the platform is in the form of a database recorded on non-transitory machine-readable storage medium comprising sequence information, length, melting temperature, and viral origin of each oligonucleotide for which sequence information was obtained.

15. A bacterial capture sequencing platform constructed by the method of claim 9 in the form of an oligonucleotide library.

16. The bacterial capture sequencing platform of claim 15, wherein the oligonucleotide library comprises oligonucleotides linked to biotin and bound to a cleavable array.

17.-28. (canceled)

29. A method of simultaneously detecting the presence of pathogenic bacteria known or suspected to infect vertebrates and/or antimicrobial resistant genes in a sample from a subject, comprising:
a. isolating nucleic acid from the sample;
b. contacting the nucleic acid with oligonucleotides of the bacterial capture sequencing platform of claim 15 to form hybridization products;
c. detecting hybridization products between the nucleic acids from the sample and the oligonucleotides;
wherein the presence of the hybridization product with an oligonucleotide originating from a particular bacterium indicates the presence of the bacterium in the sample and the presence of the hybridization product with an oligonucleotide originating from an antimicrobial resistant gene indicates the presence of the antimicrobial resistant gene in the sample.

30. The method of claim 29, wherein the sample is chosen from the group consisting of a biological sample, an environmental sample, a food sample, cells, cell culture, cell culture medium and other compositions being used for the development of pharmaceutical and therapeutic agents.

31. The method of claim 30, wherein the biological sample is chosen from the group consisting of nasopharyngeal aspirate, blood, cerebrospinal fluid, saliva, serum, urine, sputum, bronchial lavage, pericardial fluid, peritoneal fluid, feces, tissue, cells, cell culture, and cell culture medium.

32. (canceled)

33. The method of claim 29, wherein the subject is human.

34. (canceled)

35. The method of claim 29, wherein the bacterial capture sequencing platform is an oligonucleotide library.

36. A method of identifying a novel bacterium and/or antimicrobial resistant gene or biomarker in a biological sample in a sample from a subject, comprising:
a. isolating nucleic acid from the sample;
b. contacting the nucleic acid with oligonucleotides of the of the bacterial capture sequencing platform of claim 15 to form hybridization products;
c. detecting and sequencing any hybridization products between the nucleic acids from the sample and the oligonucleotides;
d. comparing the nucleotide sequence of the hybridization product to the nucleotide sequences of known bacteria and antimicrobial resistant genes; and
e. determining the bacterium and/or gene is novel if there is no identity between the sequence of the hybridization product and sequences of known bacteria and antimicrobial resistant genes.

37.-43. (canceled)

44. A method of simultaneously identifying and characterizing pathogenic bacteria and/or microbial resistance genes or biomarkers, that infect vertebrates in a sample, comprising;
a. isolating nucleic acid from the sample,
b. contacting the nucleic acid with the oligonucleotides of the bacterial capture sequencing platform of claim 15 to form hybridization products;
c. detecting and sequencing any hybridization products between the nucleic acids from the sample and the oligonucleotides;
d. comparing the nucleotide sequence of the hybridization products to the nucleotide sequences of known bacteria and/or antimicrobial genes; and
e. identifying and characterizing the bacteria by the identity between the sequence of the hybridization product and sequences of known bacteria and/or antimicrobial genes or biomarkers.

45.-59. (canceled)
